(19) United States
(12) Patent Application Publication
(10) Pub. No.: US 2023/0008595 A1
(43) Pub. Date: Jan. 12, 2023

(54) SENSOR, MEASUREMENT DEVICE PROVIDED THEREWITH, SENSOR UNIT, CELL CULTURE ANALYSIS DEVICE, AND LIQUID SAMPLE MEASUREMENT METHOD

(71) Applicant: PHC Holdings Corporation, Tokyo (JP)

(72) Inventors: Seiitirou IKETANI, Ehime (JP); Shingo OTANI, Ehime (JP); Masahiro KOUGE, Ehime (JP); Masaki YAMAMOTO, Ehime (JP); Kenta NAKAMAE, Ehime (JP); Tetsurou TAKAOKA, Ehime (JP)

(21) Appl. No.: 17/781,848

(22) PCT Filed: Nov. 12, 2020
(86) PCT No.: PCT/JP2020/042273

§ 371 Date: Jun. 2, 2022

(30) Foreign Application Priority Data
Dec. 9, 2019 (JP) 2019-222086
Dec. 25, 2019 (JP) 2019-234144

(51) Int. Cl. G01N 33/487; G01N 27/22; G01N 27/28

(52) U.S. Cl. CPC G01N 33/48707; G01N 27/22; G01N 27/28

(57) ABSTRACT

A sensor performs measurement of a culture medium and is used in a state of being immersed in a medium placed in a well, the sensor comprising a main body having a first surface and a second surface that is on the opposite side from the first surface; an electrode unit that is provided on the first surface in the main body and to which a specific voltage is applied in the course of performing measurement in a state of being immersed in the medium; and a liquid holding portion that is provided around the electrode unit on the first surface, and that is disposed near the inner wall surface of the well and holds the medium up to above the electrode unit, in between the inner wall surfaces.

### TECHNICAL FIELD

The present invention relates, for example, to a sensor for measuring a cell culture environment in a culture medium in a state of being immersed in a liquid cell medium, as well as a measurement device, a sensor unit, a cell culture analysis device, and a liquid sample measuring method equipped with this sensor.

### BACKGROUND ART

A conventional cell culture analysis device comprises a substrate, a sensor that is fixed to a through-hole portion provided to the substrate, and a lead wire that is connected to the sensor for extracting signals.

For example, Patent Literature 1 discloses a sensor that measures a cell culture environment in a medium in a state of being immersed in a liquid cell medium.

Patent Literature 2 discloses a configuration comprising an elevating mechanism (elevator) for disposing sensors on a medium.

Patent Literature 3 discloses a device and a method for analyzing cells included in a medium in a container, in which a sensor is immersed in a medium.

### CITATION LIST

### Patent Literature

Patent Literature 1: U.S. Pat. Application Publication No. 2014/0186876

Patent Literature 2: U.S. Pat. Application Publication No. 2016/0077083

Patent Literature 3: US Pat. No. 7,638,321

### SUMMARY

However, the following problem is encountered with the conventional sensor described above.

With the conventional cell culture analysis device disclosed in the above publication, a problem was that the measurement accuracy may be low because of instability of the sensor’s immersion state in the liquid sample (medium).

More specifically, near the center of the container containing the liquid sample (medium), the surface level of the liquid sample drops due to the meniscus effect.

Therefore, even if the immersion height of the sensor in the container is held constant, the electrode unit of the sensor may not be sufficiently immersed depending on the position of the sensor with respect to the container (for example, near the center).

In particular, when only a small amount of liquid sample is in the container, it is difficult to stably immerse the electrode unit of the sensor in the liquid sample.

It is an object of the present invention to improve measurement accuracy by stabilizing the immersion state of the sensor in the liquid sample.

The sensor according to the first invention is a sensor that is used in a state of being immersed in a liquid sample inside a container and that measures the liquid sample, the sensor comprising a main body, an electrode unit, and a liquid holding portion. The main body has a first surface and a second surface that is on the opposite side from the first surface. The electrode unit is provided on the first surface of the main body, and a specific voltage is applied thereto during measurement in a state of being immersed in the liquid sample. The liquid holding portion is provided around the electrode unit on the first surface, is disposed near the inner wall surface of the container, and holds the liquid sample between the inner wall surfaces.

Here, in a sensor that measures a liquid sample and is used in a state of being immersed in the liquid sample placed inside a container, an electrode unit is provided on the first surface of the main body of the sensor, and the first surface is disposed near the inner wall surface of the container, thereby forming a liquid holding portion that holds the liquid sample up to above the electrode unit, between the inner wall surface of the container and the first surface.

Here, this sensor is used in a state in which the electrode unit is immersed in the liquid sample, and is used, for example, to analyze the cell culture environment of the liquid sample.

The main body has a plate shape, for example, and has a first surface and a second surface that is on the opposite side thereof.

The electrode unit is, for example, a working electrode, a counter electrode, a reference electrode, etc., disposed on the first surface, and performs measurement of the liquid sample when voltage is applied in a state of being immersed in the liquid sample.

The electrode unit disposed on the first surface need not be provided with all of a working electrode, a counter electrode, and a reference electrode. For example, just a working electrode may be provided on the first surface. That is, one or more electrodes may be provided.

The liquid holding portion is provided on the same surface (first surface) as the electrode unit, and the first surface is disposed near the inner wall surface of the container, so that the surface tension of the liquid sample is utilized to hold the liquid sample above, so as to cover the entire electrode unit in the gap between the first surface and the inner wall surface.

Consequently, the liquid sample can be held up to a position on the first surface side disposed close to the inner wall surface of the container, that is higher than the second surface side on the opposite side from the first surface.

Therefore, it is possible to form a state in which the liquid sample is located higher on the first surface side than on the second surface side, so the electrode unit provided on the first surface can be sufficiently immersed in the liquid sample.

As a result, the sensor used in a state of being immersed in the liquid sample can measure more accurately.

The sensor according to the second invention is the sensor according to the first invention, wherein the liquid holding portion holds the liquid sample on the first surface side up to a position higher than on the second surface side when the electrode unit is immersed in the liquid sample.

Here, the liquid sample is held on the first surface side, which is disposed near the inner wall surface of the container, up to a position that is higher than on the second surface, which is on the opposite side from the first surface.

Consequently, the electrode unit provided on the first surface side is in a state of being sufficiently immersed in the liquid sample.

Therefore, the sensor used in a state of being immersed in the liquid sample can measure more accurately.

The sensor according to the third invention is the sensor according to the first or second invention, wherein the liquid holding portion is provided to the upper portion of the electrode unit on the first surface in a state in which the electrode unit is immersed in the liquid sample.

Here, the liquid holding portion for holding the liquid sample between the inner wall surface of the container and the first surface is provided to the upper portion of the electrode unit on the first surface.

Consequently, the electrode unit is disposed at a position that is lower than that of the liquid sample held by the liquid holding portion, so a state of being sufficiently immersed in the liquid sample can be formed.

The sensor according to the fourth invention is the sensor according to any of the first to third inventions, wherein the liquid holding portion has substantially the same width as the first surface, or has a width that is greater than the width of the first surface, in the portion where the electrode unit is provided.

Here, on the first surface, the width of the portion where the liquid holding portion is provided is equal to or greater than the width of the portion where the electrode unit is provided.

Consequently, it is easier to hold the liquid sample with a liquid holding portion that is wider than the portion where the electrode unit is provided.

The sensor according to the fifth invention is the sensor according to any of the first to fourth inventions, wherein the main body is disposed near the inner wall surface up to the distance at which surface tension is generated in the liquid sample held between the first surface and the inner wall surface of the container.

Here, the installation position of the main body is set so as to be disposed near and up to the distance at which surface tension is generated in the liquid sample held between the first surface and the inner wall surface of the container.

Consequently, the surface tension of the liquid sample can be utilized to hold the liquid sample on the first surface side up to a position that is higher than on the second surface.

The sensor according to the sixth invention is the sensor according to the fifth invention, wherein the container is substantially circular in top view, and the first surface is disposed at a position on a chord of the substantially circular shape with respect to the inner wall surface of the container that is substantially circular in top view.

Here, the main body (first surface) is installed so as to be disposed at a position on a chord of the substantially circular shape with respect to the substantially circular container in top view.

Consequently, the first surface of the main body can be disposed in a state of being close to the inner peripheral surface of the substantially circular container.

The sensor according to the seventh invention is the sensor according to the sixth invention, wherein the width of the first surface provided with the electrode unit is less than the diameter of the substantially circular container.

Here, the width of the portion of the first surface provided with the electrode unit is less than the diameter of the substantially circular container in top view.

Consequently, the first surface can be brought close to a position near the inner peripheral surface of the substantially circular container in top view.

The sensor according to the eighth invention is the sensor according to the fifth invention, wherein the container is substantially rectangular in top view, and the first surface is disposed so as to be near one side of the inner wall surface of the container that is substantially rectangular in top view.

Here, in top view, the main body (first surface) is installed so as to be disposed near the inner wall surface constituting one side of the substantially rectangular shape with respect to a substantially rectangular container such as a substantially square shape, for example.

Consequently, the first surface of the main body can be disposed in a state of being close to the inner wall surface of the substantially rectangular container.

The sensor according to the ninth invention is the sensor according to the eighth invention, wherein the width of the first surface provided with the electrode unit is less than the diagonal length of the substantially rectangular container.

Here, the width of the portion of the first surface where the electrode unit is provided is less than the diagonal length of the substantially rectangular container in top view.

Consequently, the first surface can be brought close to a position near the inner wall surface of the substantially rectangular container in top view.

The sensor according to the tenth invention is the sensor according to any of the first to ninth inventions, wherein the electrode unit includes at least one of a reference electrode, a working electrode, and a counter electrode.

Here, an electrode unit including at least one of a reference electrode, a working electrode, and a counter electrode is used.

Consequently, the liquid sample can be measured by applying voltage while each electrode is immersed in the liquid sample.

The sensor according to the eleventh invention is the sensor according to any of the first to tenth inventions, wherein the main body is substantially L-shaped or substantially inverted T-shaped in front view.

Here, a sensor comprising a main body that is substantially L-shaped or substantially inverted T-shaped is used.

Consequently, the liquid holding portion disposed in the portion including the electrode unit immersed in the liquid sample, on the upper portion thereof, etc., can be provided to a portion that is wide and is substantially L-shaped or substantially inverted T-shaped.

As a result, the electrode unit can be sufficiently immersed in the liquid sample in a state of holding the liquid sample in the wide portion.

The sensor unit according to the twelfth invention comprises a sensor according to any of the first to eleventh inventions, a substrate provided with a plurality of sensors, and a connection portion for connecting the substrate and the sensors.

Here, the plurality of sensors are connected to the substrate via the connection portion.

Consequently, a plurality of sensors can be simultaneously immersed in a plurality of containers by lowering the substrates disposed on the upper parts of a plurality of containers.

The sensor unit according to the thirteenth invention is the sensor unit according to the twelfth invention, wherein the plurality of sensors are formed by cutting out a part of the substrate.

Here, a part of the substrate is cut out to form a plurality of sensors.

Consequently, by cutting out a part of the substrate and bending it to form a sensor, it is possible to obtain a configuration in which a plurality of sensors are included in a single substrate.

The sensor unit according to the fourteenth invention is the sensor unit according to the twelfth or thirteenth invention, further comprising a bottom cover provided below the substrate, and a top cover provided above the substrate, wherein the substrate is sandwiched between the bottom cover and the top cover from above and below.

Consequently, it possible to configure a sensor unit in which a substrate including a plurality of sensors is disposed so as to be sandwiched between the bottom cover and the top cover from above and below.

The sensor unit according to the fifteenth invention is the sensor unit according to the fourteenth invention, wherein the bottom cover is provided with through-holes through which the sensors pass downward.

Here, a bottom cover is used in which through-holes are formed through which the plurality of sensors included in the substrate pass downward.

Consequently, a plurality of sensors can be immersed through the through-holes into a plurality of containers disposed below the bottom cover.

The cell culture analysis device according to the sixteenth invention comprises the sensor unit according to any of the twelfth to fifteenth inventions, and a culture container installation unit on which the sensor unit is placed.

Here, a cell culture analysis device is configured to include the above-mentioned sensor unit and a culture container installation unit on which are placed the sensor unit and a culture container containing a liquid sample.

Consequently, the various effects mentioned above can be obtained by disposing the sensors of the sensor unit close to the inner wall surface of the culture container installed in the culture container installation unit.

The sensor according to the seventeenth invention is used in a state of being immersed in a liquid sample inside a container, and measures the cell culture environment in the liquid sample, the sensor comprising a main body and an electrode unit. The main body has a first surface and a second surface that is on the opposite side from the first surface. The electrode unit is provided on the first surface of the main body, and a specific voltage is applied thereto during measurement in a state of being immersed in the liquid sample. In carrying out a measurement, the main body is installed at a position that is offset from the center of the container.

Here, the sensor is used in a state of being immersed in a liquid sample inside a container and measures the liquid sample, an electrode unit is provided on the first surface of the main body of the sensor, and the main body is installed at a position that is offset from the center of the container.

Here, this sensor is used in a state in which the electrode unit is immersed in the liquid sample, and is used, for example, to analyze the cell culture environment of the liquid sample.

The main body plate-shaped, for example, has a first surface and a second surface on the opposite side thereof, and is installed at a position that is offset from the center of the container containing the liquid sample.

The electrode unit is, for example, a reference electrode, a counter electrode, a working electrode, etc., disposed on the first surface, and measures the liquid sample when voltage is applied in a state of being immersed in the liquid sample.

Consequently, the sensor is disposed at a position that is offset from the center of the container, that is, a position near the inner wall surface. Therefore, on the first surface of the main body disposed close to the inner wall surface, the liquid sample can be held at a position that is higher than on the second surface side, which is on the opposite side from the first surface.

Therefore, on the first surface side, it is possible to form a state in which the liquid sample is located higher than on the second surface side, so the electrode unit provided on the first surface can be sufficiently immersed in the liquid sample.

As a result, the sensor used in a state of being immersed in the liquid sample can measure more accurately.

The sensor according to the eighteenth invention is a sensor that is used in a state of being immersed in a liquid sample inside a container, and that measures the liquid sample, the sensor comprising a main body, a measurement electrode unit, and an immersion detection electrode unit. The measurement electrode unit is provided to the main body, and a specific first voltage is applied thereto during measurement in a state of being immersed in the liquid sample. The immersion detection electrode unit is provided above the measurement electrode unit in the main body in a state of being immersed in the liquid sample, and a specific second voltage is applied thereto during detection of whether or not the measurement electrode unit is in a state of being immersed in the liquid sample.

Here, the sensor is used in a state of being immersed in a liquid sample placed in a container and measures the liquid sample, a measurement electrode unit and a immersion detection electrode unit are provided to the main body of the sensor, and a specific voltage (second voltage) is applied to the immersion detection electrode unit provided above the measurement electrode unit to detect the immersion state of the measurement electrode unit.

Here, this sensor is used in a state in which the measurement electrode unit is immersed in the liquid sample, and is used, for example, to analyze the cell culture environment of the liquid sample.

The main body is plate shaped, for example, and the measurement electrode unit and the immersion detection electrode unit are provided on the same or different surfaces thereof.

The measurement electrode unit is, for example, a working electrode, a counter electrode, a reference electrode, etc., disposed in the main body, and measures the concentration or the like of the liquid sample when voltage is applied in a state of being immersed in the liquid sample.

The measurement electrode unit disposed on the main body does not need to have all of the above-mentioned working electrode, counter electrode, and reference electrode on the same surface. For example, the configuration may be such that a working electrode is provided on the first surface and another electrode is provided on the second surface.

The immersion detection electrode unit is, for example, one to four electrodes provided to the main body in order to detect whether or not the measurement electrode unit is sufficiently immersed in the liquid sample. The immersion detection electrode unit is disposed at a position above the measurement electrode unit. For example, it is disposed with its lower end portion substantially flush with, or slightly above, the upper end portion of the measurement electrode unit.

Here, the first voltage applied to the measurement electrode unit when measuring the liquid sample and the second voltage applied to the immersion detection electrode unit when detecting the immersion state of the measurement electrode unit may both be the same voltage, or may be different voltage values.

Also, the measurement of the liquid sample and the detection of the immersion state of the measurement electrode unit may be performed at different points in time, or may be performed at the same time.

Consequently, when the first voltage is applied to the measurement electrode unit to measure the liquid sample, the second voltage is applied to the immersion detection electrode unit, and the immersion state of the measurement electrode unit, that is, whether or not the measurement electrode unit is immersed in the liquid sample, can be accurately detected.

Consequently, the measurement electrode unit can reliably perform measurement in a state of being immersed in the liquid sample by taking measures according to the detected immersion state of the measurement electrode unit, such as adding more liquid sample to the container or moving the position of the sensor in the immersion depth direction.

As a result, the measurement accuracy can be improved by detecting the immersion state of the measurement electrode unit of the sensor in the liquid sample.

The sensor according to the nineteenth invention is the sensor according to the eighteenth invention, wherein the main body has a first surface to which the measurement electrode unit is provided. The immersion detection electrode unit is disposed above the measurement electrode unit on the first surface.

Here, the measurement electrode unit and the immersion detection electrode unit are provided on the same surface (first surface) of the main body.

Consequently, whether or not the measurement electrode unit is immersed in the liquid sample can be detected accurately by using the immersion detection electrode unit provided on the same surface as the measurement electrode unit to detect the immersion state of the measurement electrode unit in the liquid sample.

The sensor according to the twentieth aspect of the invention is the sensor according to the eighteenth or nineteenth invention, further comprising a protective film that covers at least a part of the measurement electrode unit.

Here, a protective film is provided to cover a reagent disposed on an electrode, such as a working electrode, included in the measurement electrode unit, for example.

Consequently, the permeation rate of a specific component in the liquid sample, and the rate at which the reagent flows out into the liquid sample can be controlled by the protective film covering at least part of the measurement electrode unit.

The sensor according to the twenty-first invention is the sensor according to any of the eighteenth to twentieth inventions, wherein the measurement electrode unit includes two poles, namely, a working electrode and a counter electrode, or includes three poles, namely, a working electrode, a counter electrode, and a reference electrode.

Here, a two-pole configuration of a working electrode and a counter electrode, or a three-pole configuration of a working electrode, a counter electrode, and a reference electrode is employed as the measurement electrode unit.

Consequently, the concentration and the like of the liquid sample can be measured by using a measurement electrode unit including either two electrodes or three electrodes.

The sensor according to the twenty-second invention is the sensor according to the nineteenth invention, wherein the protective film is provided so as to cover at least the working electrode included in the measurement electrode unit.

Here, a protective film is provided on the working electrode, which tends to have a greater effect on measurement accuracy when measuring a liquid sample.

Consequently, even in a configuration in which a reagent is provided on a working electrode, for example, the outflow of the reagent to the liquid sample and the permeation rate of a specific component in the liquid sample can be controlled by the protective film.

The sensor according to the twenty-third invention is the sensor according to any of the eighteenth to twenty-second inventions, wherein the immersion detection electrode unit is disposed directly on the working electrode included in the measurement electrode unit.

Here, the immersion detection electrode unit is provided directly on the working electrode, which tends to have a greater effect on measurement accuracy when measuring a liquid sample.

Consequently, among the electrodes included in the measurement electrode unit, the immersion detection electrode unit is provided directly on the working electrode that tends to have a greater effect on measurement accuracy, so the immersion state of at least the working electrode can be accurately detected.

The sensor according to the twenty-fourth invention is the sensor according to any of the eighteenth to the twenty-third inventions, wherein the immersion detection electrode unit is installed so as to match the width of the working electrode included in the measurement electrode unit in a substantially horizontal direction.

Here, the immersion detection electrode unit is disposed to match the width of the working electrode (the dimension in the substantially horizontal direction).

Consequently, among the electrodes included in the measurement electrode unit, the immersion detection electrode unit is provided to match the width of the working electrode, which tends to have a greater effect on measurement accuracy, so the immersion state of at least the working electrode can be accurately detected.

The sensor according to the twenty-fifth invention is the sensor according to any of the eighteenth to twenty-fourth inventions, wherein the immersion detection electrode unit has two or three electrodes.

Here, the immersion detection electrode unit is constituted by two or three electrodes.

Consequently, whether or not the measurement electrode unit is sufficiently immersed in the liquid sample can be detected by applying voltage between two electrodes, or by applying voltage to two out of three electrodes, and measuring the current value.

The sensor according to the twenty-sixth invention is the sensor according to any of the eighteenth to twenty-fifth inventions, wherein the immersion detection electrode unit has one electrode. The second voltage is applied between the one electrode and at least one of the electrodes constituting the measurement electrode unit.

Here, one electrode and an electrode included in the measurement electrode unit are used as the immersion detection electrode unit.

Consequently, the immersion state of the measurement electrode unit can be detected, and the liquid sample can be measured, with a simple configuration.

The sensor according to the twenty-seventh invention is the sensor according to any of the eighteenth to twenty-sixth inventions, wherein the immersion detection electrode unit extends in the form of a plurality of comb-like teeth substantially parallel to the surface level of the liquid sample.

Here, an electrode in the form of a plurality of comb-like teeth is used as the immersion detection electrode unit.

Consequently, any change in the immersion state in the depth direction of the liquid sample can be amplified and more easily detected by the plurality of comb teeth extending substantially parallel to the surface level of the liquid sample. Therefore, not only whether or not the measurement electrode unit is immersed, but also the height of the surface level of the liquid sample in the container can also be detected.

The sensor according to the twenty-eighth invention is the sensor according to any of the eighteenth to twenty-seventh inventions, wherein the immersion detection electrode unit has a shape whose dimension in the substantially horizontal direction changes in the immersion depth direction in a state of being immersed in the liquid sample.

Here, an electrode whose dimensions change in the substantially horizontal direction, such as an electrode having a substantially triangular shape, is used as the immersion detection electrode unit.

Consequently, any change in the surface level of the liquid sample can be amplified and more easily detected, so not only whether or not the measurement electrode unit is immersed, but also the height of the surface level of the liquid sample in the container can be detected.

The sensor according to the twenty-ninth invention is the sensor according to the twenty-eighth invention, wherein the immersion detection electrode unit has a substantially triangular shape.

Here, an electrode having a substantially triangular shape is used as the immersion detection electrode unit.

Consequently, by disposing a substantially triangular electrode so that its apex faces upward or downward, a change in the height of the surface level of the liquid sample can be amplified and more easily detected, so not only whether or not the measurement electrode unit is immersed, but also the height of the surface level of the liquid sample in the container can be detected.

The sensor according to the thirtieth invention is the sensor according to any of the eighteenth to the twenty-ninth inventions, wherein the working electrode included in the measurement electrode unit is disposed at a position in the main body that is away from the counter electrode included in the measurement electrode unit.

Here, the working electrode included in the measurement electrode unit is provided at a position that is away from the counter electrode in the main body.

Consequently, even when a protective film is provided so as to cover only the working electrode, for example, since this protective film will be disposed at a position that is away from the counter electrode, it can be easily provided on the working electrode.

The sensor according to the thirty-first invention is the sensor according to any of the eighteenth to thirtieth inventions, wherein the immersion detection electrode unit is disposed in the approximate center portion of the container.

Here, the immersion detection electrode unit for detecting the immersion state of the measurement electrode unit is disposed near the approximate center of the container.

Here, in the liquid sample inside the container, the area near the inner wall surface of the container is lifted by the meniscus effect, and the surface level tends to be the lowest near the center of the container.

Therefore, in this sensor, the immersion detection electrode unit is disposed near the center of the container where the surface level is assumed to be the lowest.

Consequently, no matter where in the container the measurement electrode unit is disposed, whether or not it is in an immersed state can be accurately detected by detecting whether there is any liquid sample near the center of the container where the surface level tends to be the lowest.

The measurement device according to the thirty-second invention comprises the sensor according to any of the eighteenth to thirty-first claims, a voltage application unit that applies a specific first voltage and second voltage to the measurement electrode unit and the immersion detection electrode unit, and a control unit that performs measurement on the liquid sample on the basis of a first current value obtained by applying the first voltage to the measurement electrode unit, and detects whether or not the measurement electrode unit is in an immersed state on the basis of a second current value obtained by applying the second voltage to the immersion detection electrode unit.

Here, the control unit measures the liquid sample and detects the immersion state of the measurement electrode unit on the basis of the current values (first current value and second current value) detected upon application of specific voltages (first voltage and second voltage) from the voltage application unit to the measurement electrode unit and the immersion detection electrode unit of the sensor discussed above.

Consequently, it is possible to configure a measurement device that measures a liquid sample and detects the immersion state of a measurement electrode on the basis of a first current value and a second current value.

The measurement device according to the thirty-third invention is the measurement device according to the thirty-second invention, wherein the control unit detects the surface level of the liquid sample on the basis of the second current value obtained by applying the second voltage to the immersion detection electrode unit.

Here, the surface level of the liquid sample in the container is detected on the basis of the change in the second current value detected when the second voltage is applied to the immersion detection electrode unit.

Here, it is preferable to use, for example, an electrode that is longer in the immersion depth direction, a comb-shaped electrode, a substantially triangular electrode whose dimensions change in the substantially horizontal direction, or the like as the immersion detection electrode unit.

Consequently, in addition to the immersion state of the measurement electrode unit, the surface level height of the liquid sample in the container can also be detected.

The measurement device according to the thirty-fourth invention is the measurement device according to the thirty-second or thirty-third invention, wherein the voltage application unit applies a substantially AC voltage to the measurement electrode unit and the immersion detection electrode unit.

Here, a substantially AC voltage is used as the voltage applied to the measurement electrode unit and the immersion detection electrode unit.

Here, the “substantially AC voltage” includes, for example, a voltage having a sine waveform, a voltage having a square waveform, and so forth.

Consequently, a higher current value can be obtained than when DC voltage is applied, so the S/N ratio and the detection accuracy can be improved, for example.

The sensor unit according to the thirty-fifth invention comprises the sensor according to any of the eighteenth to thirty-first inventions, a substrate provided with a plurality of the sensors, and a connection portion that connects the substrate and the sensors.

Here, a plurality of sensors are connected to the substrate via the connection portion.

Consequently, it is possible to immerse a plurality of sensors in a plurality of containers all at the same time by lowering a substrate disposed at the upper part of the plurality of containers.

The sensor unit according to the thirty-sixth invention is the sensor unit according to the thirty-fifth invention, wherein the plurality of sensors are formed by cutting out parts of the substrate.

Here, parts of the substrate are cut out to form a plurality of sensors.

Consequently, it is possible to obtain a configuration in which a plurality of sensors are included on a single substrate by cutting out parts of the substrate and bending them to form sensors.

The sensor unit according to the thirty-seventh invention is the sensor unit according to the thirty-fifth or thirty-sixth invention, further comprising a bottom cover provided below the substrate, and a top cover provided above the substrate. The substrate is configured to be sandwiched between the bottom cover and the top cover from above and below.

This results in a sensor unit configuration in which a substrate including a plurality of sensors is disposed so as to be sandwiched between a bottom cover and a top cover from above and below.

The sensor unit according to the thirty-eighth invention is the sensor unit according to the thirty-seventh invention, wherein the bottom cover is provided with through-holes through which the sensors pass downward.

Here, through-holes are formed in the bottom cover so that the plurality of sensors included in the substrate pass downward through the holes.

Consequently, the plurality of sensors can be immersed through the through-holes into the plurality of containers disposed under the bottom cover.

The sensor unit according to the thirty-ninth invention is a sensor unit comprising a plurality of sensors that are used in a state of being immersed in a liquid sample contained in a plurality of containers, the sensor unit further comprising a first sensor and a second sensor. The first sensor is provided at a position corresponding to a first container disposed on at least one edge, out of the plurality of containers. The second sensor is provided at a position corresponding to a second container disposed at a position other than that of the first sensor, out of the plurality of containers. The first sensor has an immersion detection electrode unit for detecting whether or not the measurement electrode of the second sensor immersed in the liquid sample is itself immersed in the liquid sample. The second sensor has a measurement electrode unit for measuring the liquid sample.

Here, regarding the sensors immersed in the liquid sample contained in a plurality of containers, a first sensor having an immersion detection electrode unit for detecting whether or not the measurement electrode of the second sensor immersed in the liquid sample is itself immersed in the liquid sample is used as a sensor disposed at a position corresponding to a container disposed on at least one edge, and a second sensor having a measurement electrode unit for measuring a liquid sample is used as a sensor disposed at a position corresponding to a container disposed at a position other than that of the first sensor.

Here, the first sensor may be configured to have not only an immersion detection electrode unit but also a measurement electrode unit. Also, the second sensor may be configured to have not only a measurement electrode unit but also an immersion detection electrode unit.

Consequently, the first sensor having the immersion detection electrode unit can be disposed at a position corresponding to a container disposed at a position where the surface level of the liquid sample is most prone to dropping through evaporation, out of the plurality of disposed containers.

As a result, the immersion state of the measurement electrode unit in another container can be easily recognized by detecting the surface level of the liquid sample in a container disposed at a position where the liquid sample is most prone to dropping through evaporation.

Also, when a configuration without a measurement electrode unit is employed as the first sensor, and a configuration without an immersion detection electrode unit is employed as the second sensor, sensors with a simple configuration can be used to detect the immersion state of the measurement electrode units in a plurality of containers.

The sensor unit according to the fortieth invention is the sensor unit according to the thirty-ninth invention, wherein the first sensors are disposed at positions corresponding to the containers disposed at the four corners, among a plurality of containers disposed in a substantially rectangular shape.

Here, for example, among a plurality of containers disposed in a substantially rectangular shape, such as a substantially oblong rectangular shape, first sensors having an immersion detection electrode unit are disposed at positions corresponding to the containers disposed at the four corners.

Consequently, it is possible to detect only the surface level by using the containers at the four corners, where the liquid sample is most prone to dropping through evaporation, as dummy containers.

The cell culture analysis device according to the forty-first invention comprises the sensor unit according to any of the thirty-fifth to fortieth claims, and a culture container installation unit on which are placed the sensor unit and the container in which the liquid sample is contained.

Here, a cell culture analysis device is configured to include the sensor unit and a culture container installation unit on which are placed the sensor unit and a culture container containing a liquid sample.

Consequently, the various effects mentioned above can be obtained by disposing the sensor of the sensor unit in the container installed in the culture container installation unit.

The cell culture analysis device according to the forty-second invention is the cell culture analysis device according to the forty-first invention, further comprising an immersion detection unit that is connected to the immersion detection electrode units of the plurality of sensors provided on the substrate, and that detects the immersion state of the measurement electrode unit with respect to the liquid sample contained in the container, and a display unit that displays the detection result from the immersion detection unit.

Here, the immersion state of the measurement electrode unit detected by the plurality of sensors is displayed by using a display unit that lights a lamp, for example.

Consequently, if it is detected that the measurement electrode unit is sufficiently immersed in the liquid sample, the lamp of the display unit connected to each sensor can be lit, for example.

As a result, the user can easily recognize whether the measurement electrode unit of a sensor is in an immersed or unimmersed state, and in which container, and can care measures such as adding more liquid sample to any containers in which the light is not lit because of an unimmersed state.

The liquid sample measuring method according to the forty-third invention is a liquid sample measurement method for measuring with the sensor according to the eighteenth to thirty-first inventions, the method comprising an immersion detection step of applying the second voltage to the immersion detection electrode unit, and a measurement step of applying the first voltage to the measurement electrode unit.

Here, in the above-mentioned measurement method in which a liquid sample is measured using a sensor, a second voltage is applied to the immersion detection electrode unit to detect the immersion state of the measurement electrode unit in the liquid sample, and then a first voltage is applied to the measurement electrode unit to measure the concentration, etc., of the liquid sample.

Consequently, it is possible to detect whether or not the measurement electrode is sufficiently immersed in the liquid sample before the liquid sample is measured, so measurement accuracy can be improved.

The liquid sample measuring method according to the forty-fourth invention is the liquid sample measuring method according to the forty-third invention, further comprising a voltage application stoppage step of stopping the application of the second voltage to the immersion detection electrode unit, in between the immersion detection step and the measurement step.

Here, a period of no voltage application is provided in between the detection of the immersion state of the measurement electrode unit and the measurement of the liquid sample.

Consequently, the voltage applied for immersion detection is less likely to affect the measurement of the liquid sample, which allows the measurement of the liquid sample to be performed more accurately.

### EFFECTS

With the sensor according to the present invention, the measurement accuracy of a sensor used in a state of being immersed in a liquid sample can be improved.

### BRIEF DESCRIPTION OF THE DRAWINGS

FIG. 1 is a front view of a cell culture device in which is installed a cell culture analysis device comprising a sensor unit according to an embodiment of the present invention;

FIG. 2 is an oblique view of the cell culture device of FIG. 1;

FIG. 3 is an oblique view of the cell culture analysis device of FIG. 1;

FIG. 4 is an oblique view of the cell culture analysis device of FIG. 1;

FIG. 5 is an exploded oblique view of the cell culture analysis device of FIG. 1;

FIG. 6 is a control block diagram of the cell culture analysis device of FIG. 1;

FIG. 7 is a detail oblique view of the sensor unit of FIG. 1;

FIG. 8 is an exploded oblique view of the sensor unit of FIG. 1;

FIG. 9 is a partially cut-away oblique view of the sensor unit of FIG. 1;

FIG. 10 is a detail cross-sectional view of the sensor unit of FIG. 1;

FIG. 11 is a detail plan view of the sensor unit of FIG. 1;

FIG. 12 is a detail oblique view of the sensor unit of FIG. 1;

FIG. 13 is a front view of the sensor included in the sensor unit of FIG. 1;

FIG. 14 is a top view showing a state in which the sensor of FIG. 13 is installed in a well;

FIG. 15 is a side view of FIG. 14;

FIG. 16 is a front view showing the configuration of a sensor according to another embodiment of the present invention;

FIG. 17 is a front view showing the configuration of a sensor according to yet another embodiment of the present invention;

FIG. 18 is a top view showing a state in which a sensor is inserted into a well according to yet another embodiment of the present invention;

FIG. 19 is an oblique view of a cell culture analysis device according to another embodiment of the present invention;

FIG. 20 is an oblique view of the cell culture analysis device of FIG. 19;

FIG. 21 is an exploded oblique view of the cell culture analysis device of FIG. 19;

FIG. 22 is a control block diagram of the cell culture analysis device of FIG. 19;

FIG. 23 is a detail oblique view of the sensor unit of FIG. 19;

FIG. 24 is an exploded oblique view of the sensor unit of FIG. 19;

FIG. 25 is a partially cut-away oblique view of the sensor unit of FIG. 19;

FIG. 26 is a detail cross-sectional view of the sensor unit of FIG. 19;

FIG. 27 is a partial plan view of the sensor unit of FIG. 19;

FIG. 28 is a detail oblique view of the sensor unit of FIG. 19;

FIG. 29 is a front view of the sensor included in the sensor unit of FIG. 19;

FIG. 30 is a top view showing a state in which the sensor of FIG. 29 is installed in a well;

FIG. 31 is a front view showing an immersion state in which the sensor of FIG. 29 is installed in a well;

FIG. 32 is a control block diagram showing a configuration in which voltage is applied to an immersion detection electrode unit included in the sensor of FIG. 29;

FIG. 33 is a circuit diagram showing the configuration of the control unit of FIG. 29;

FIG. 34A is a diagram showing a square wave applied by the control unit of FIG. 33, and FIG. 34B is a graph of the current value detected by applying voltage in FIG. 34A;

FIGS. 35A and 35B are diagrams showing an unimmersed state in which the sensor of FIG. 29 is installed in a well;

FIG. 36 is a flowchart showing the flow in a method for measuring a liquid sample using the sensor of FIG. 29;

FIG. 37 is a front view showing the configuration of a sensor according to another embodiment of the present invention;

FIG. 38 is a circuit diagram showing the circuit configuration of the sensor of FIG. 37;

FIG. 39 is a front view showing the configuration of a sensor according to yet another embodiment of the present invention;

FIG. 40 is a circuit diagram showing the circuit configuration of the sensor of FIG. 39;

FIG. 41 is a front view showing the configuration of a sensor according to yet another embodiment of the present invention;

FIG. 42 is a circuit diagram showing the circuit configuration of the sensor of FIG. 41;

FIG. 43 is a front view showing the configuration of a sensor according to yet another embodiment of the present invention;

FIGS. 44A to 44D are front views showing the detection of the surface level using the sensor of FIG. 43;

FIG. 45 is a graph of the value of the detected current corresponding to a change in the surface level in FIGS. 44A to 44D;

FIG. 46 is a front view showing the configuration of a sensor according to yet another embodiment of the present invention;

FIGS. 47A to 47C are graphs of the change in the detected current value according to the shape of the electrode included in the sensor;

FIG. 48 is a front view showing the configuration of a sensor according to yet another embodiment of the present invention;

FIG. 49 is a front view showing the configuration of a sensor according to yet another embodiment of the present invention;

FIG. 50A is a front view showing the configuration of a sensor according to yet another embodiment of the present invention, and FIG. 50B is a diagram of a configuration in which the sensor of FIG. 50A has been unitized;

FIGS. 51A and 51B are front views showing the configuration of a sensor according to yet another embodiment of the present invention;

FIG. 52A is a front view showing the configuration of a sensor including an immersion detection electrode unit included in a sensor unit according to yet another embodiment of the present invention, and FIG. 52B is a plan view showing the positions of wells in which the sensor of FIG. 50A is installed;

FIG. 53 is a front view showing a state in which the immersion detection electrode unit of the sensor according to yet another embodiment of the present invention is disposed near the center of the well;

FIG. 54 is a front view showing the configuration of a sensor according to yet another embodiment of the present invention; and

FIGS. 55A and 55B are diagrams of the shape of the main body of the sensor according to yet another embodiment of the present invention.

### DESCRIPTION OF EMBODIMENTS

### Embodiment 1

A sensor 16 according to an embodiment of the present invention, a sensor unit 9 comprising this sensor 16, and a cell culture analysis device 3 will now be described with reference to the appended drawings.

### Overview of Cell Culture Device 1

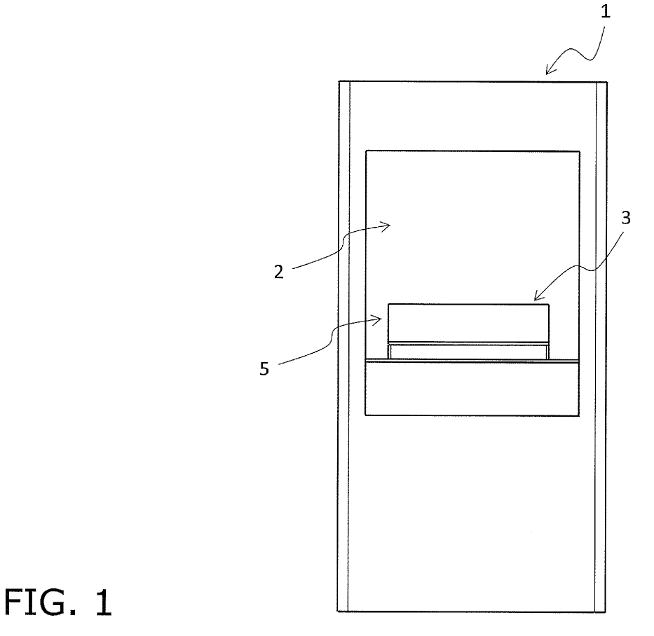

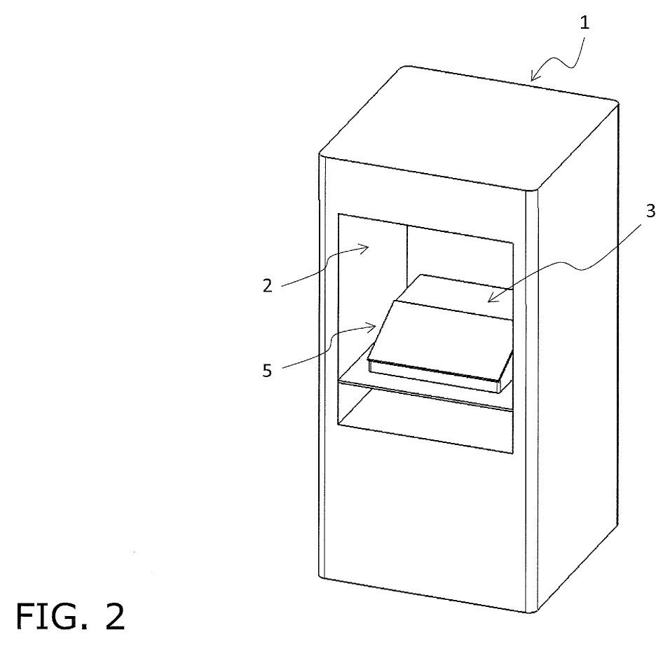

As shown in FIGS. 1 and 2, a cell culture device 1 comprises a culture chamber 2 and the cell culture analysis device 3.

The cell culture analysis device 3 is disposed in the culture chamber 2 of the cell culture device 1. Although not shown in FIGS. 1 and 2, a door is attached to the front of the culture chamber 2 so as to allow the door to be opened and closed. In the culture chamber 2, cell culture is performed, and the state of the cell culture environment is detected using the sensors 16 of the cell culture analysis device 3 (discussed below).

### Cell Culture Analysis Device 3

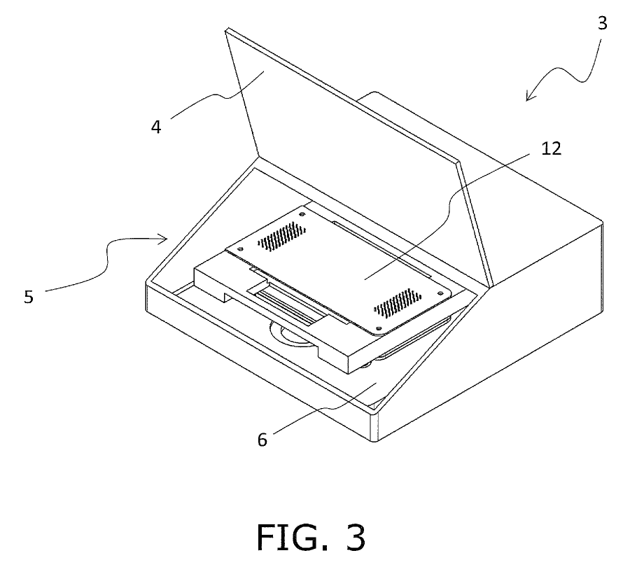

As shown in FIG. 3, the cell culture analysis device 3 comprises a door 4, a main body case 5, and a culture container installation unit 6.

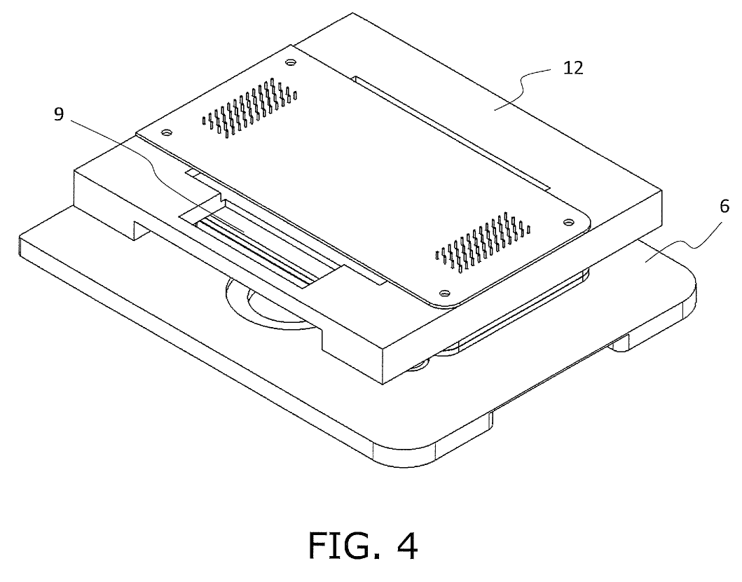

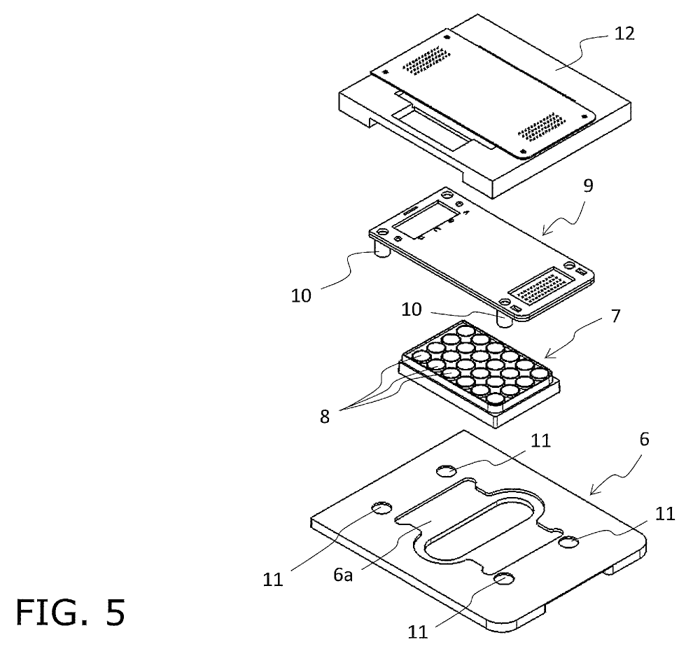

As shown in FIG. 3, the culture container installation unit 6 is disposed in the main body case 5 provided with the door 4 on its front side. As shown in FIGS. 4 and 5, a culture container 7 and a sensor unit 9 are placed on the culture container installation unit 6.

As shown in FIG. 5, the culture container 7 has 24 wells (containers) 8, for example. Each of the wells 8 contains a liquid culture medium (liquid sample) to be analyzed using the sensor unit 9.

The wells 8 are substantially cylindrical containers having a diameter of 15.1 mm, for example, into which is inserted a sensor 16 having a width of about 7.0 mm. From 0.5 to 1.0 mL of medium (liquid sample) is placed in each well 8, for example.

Also, the culture container 7 is installed in a state of being positioned in a substantially rectangular recess 6a formed in the cell culture installation unit 6. The recess 6a is a recessed portion formed to match the external shape of the culture container 7, has substantially the same external shape as the culture container 7, and holds the culture container 7 so as not to move in the plane direction.

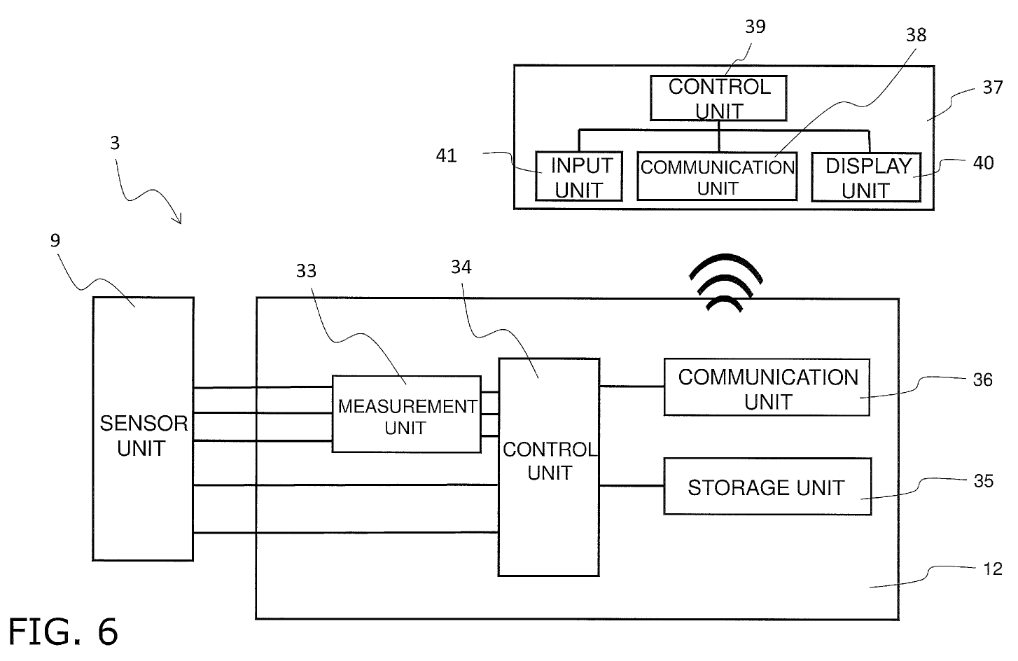

FIG. 6 shows the control blocks of the cell culture analysis device 3. That is, as shown in FIG. 6, the cell culture analysis device 3 comprises a control unit 12 including a measurement unit 33 and a control unit 34 (which are connected to the sensor unit 9), a storage unit 35, and a communication unit 36.

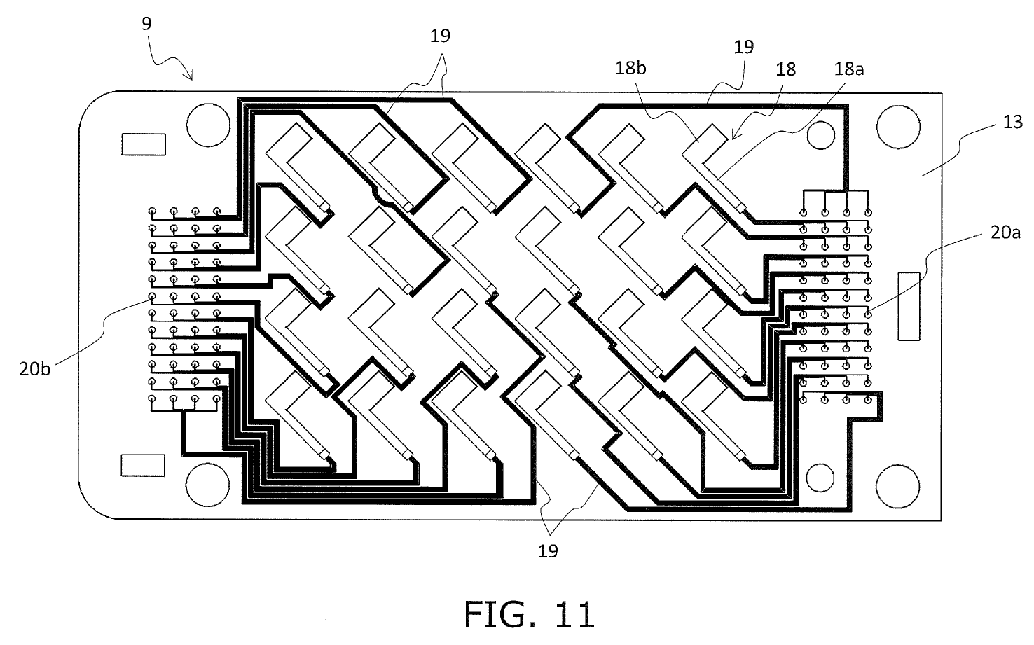

The control unit 12 applies voltage to the electrode unit 21 of each sensor 16 included in the sensor unit 9, via connection portions 20a and 20b (see FIG. 11). Then, the control unit 12 transmits information about the cell culture environment of the medium (liquid sample) contained in the wells 8 to a data processing device (such as a personal computer) external to the cell culture device 1.

Information about the cell culture environment in the wells 8 detected by the sensors 16 is transmitted to the control unit 34 via the measurement unit 33 provided inside the control unit 12, and is stored in the storage unit 35. The information about the cell culture environment stored in the storage unit 35 is then transmitted to the communication unit 38 of an external device 37 (such as a personal computer) via the communication unit 36.

The external device 37 comprises a communication unit 38, a control unit 39, a display unit 40, and an input unit 41 (such as a mouse or a keyboard). In the external device 37, the control unit 39 controls the display unit 40 so that the display unit 40 displays the detected data.

### Sensor Unit 9

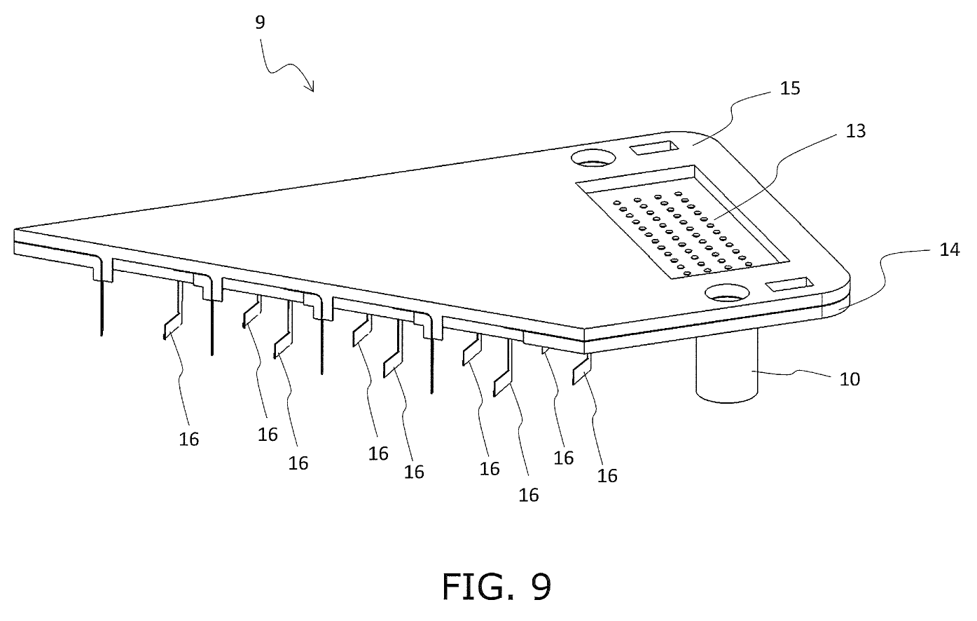

As shown in FIG. 5, the sensor unit 9 including the sensors 16 (see FIG. 9) for analyzing the state of the cell culture environment of the medium (liquid sample) contained in the wells 8 is disposed over the culture container 7.

As shown in FIG. 5, the sensor unit 9 has four legs (support portions) 10 provided on its lower surface, and these legs are inserted into positioning holes 11 provided in the culture container installation unit 6. Consequently, the sensor unit 9 is installed at a specific position in the culture container 7 in a state of being separated from the culture container installation unit 6 by a specific distance.

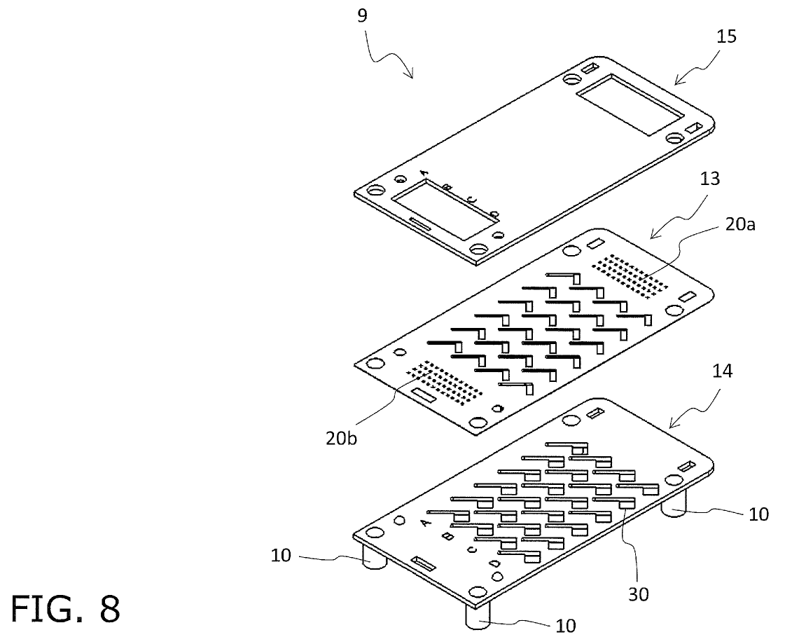

That is, as shown in FIG. 8, the legs 10 for ensuring a space for accommodating the plurality of wells 8 included in the culture container 7 are provided on the lower surface side of the sensor unit 9, over the culture container installation unit 6. The sensor unit 9 is disposed on the culture container installation unit 6 by means of the legs 10.

As described above, the legs 10 support the sensor unit 9 on the culture container installation unit 6 with a specific gap in between in order to ensure enough space for the culture container 7 over the culture container installation unit 6.

Here, the support portions that support the sensor unit 9 from below are not limited to legs that are provided to the sensor unit 9. For example, these may be any supports that support the sensor unit 9 from below with respect to the culture container installation unit 6.

Also, as shown in FIG. 5, the above-mentioned control unit 12 is disposed on the sensor unit 9.

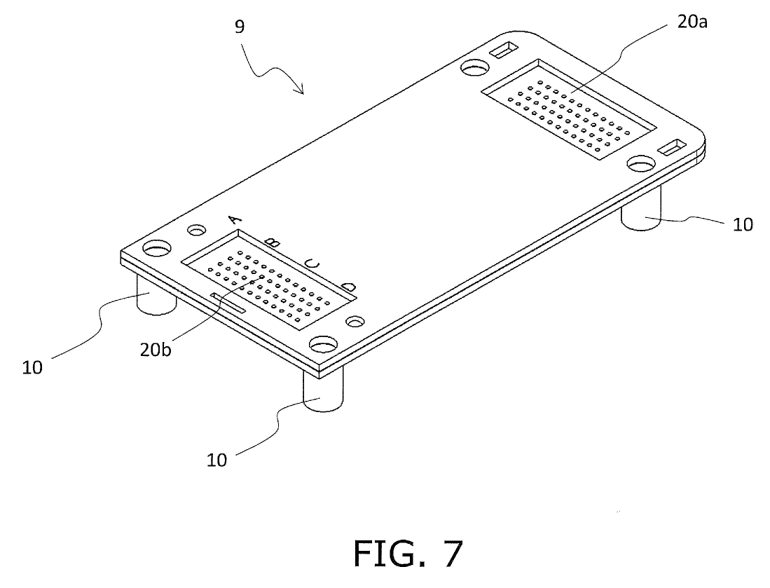

As shown in FIGS. 7 to 9, the sensor unit 9 comprises, for example, a substrate 13 molded from PET (polyethylene terephthalate; a resin material), a bottom cover 14 disposed below the substrate 13, and a top cover 15 disposed above the substrate 13. The substrate 13 is sandwiched from above and below by the bottom cover 14 and the top cover 15.

As shown in FIG. 9, the substrate 13 is provided with a plurality of sensors 16. More specifically, the sensors 16 are formed by cutting out portions of the substrate 13, leaving on the substrate 13 bent portions 17 produced by the downward bending of the connection portions between the sensors 16 and the substrate 13.

As shown in FIG. 9, the substrate 13 to which the sensors 16 are connected is sandwiched between the top cover 15 and the bottom cover 14 from above and below. As shown in FIG. 8, the bottom cover 14 is provided with a plurality of through-holes 30. Consequently, the lateral side portions of the sensors 16 (the portions where the working electrodes 21a, the counter electrodes 21b, and the reference electrodes 21c are located) pass through the through-holes 30 and are disposed below the bottom cover 14.

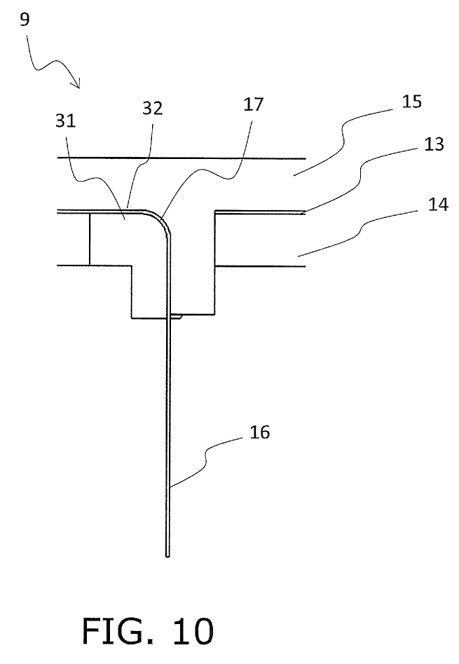

In this embodiment, as shown in FIG. 10, a support portion 31 that supports the lower side of the bent portion 17 of each sensor 16 is provided at the opening edge of each through-hole 30 in the bottom cover 14. A pressing portion 32 that pushes the upper side of the bent portion 17 of the sensor 16 downward is provided at the portion of the top cover 15 that is opposite the support portion 31.

These support portions 31 have a curved upper surface shape. Also, the pressing portions 32 have a curved lower surface shape.

Therefore, as shown in FIGS. 9 and 10, when the substrate 13 is sandwiched between the top cover 15 and the bottom cover 14 from above and below, the bent portions 17 of the sensors 16 are sandwiched from above and below by the support portions 31 and the pressing portions 32.

Consequently, the lateral side portions of the substantially L-shaped sensors 16 (the portion provided with the working electrode 21a, the counter electrode 21b, and the reference electrode 21c) can be stably maintained in a state of being disposed along the substantially horizontal direction.

Thus, the lateral side portions of the sensors 16 (the portion provided with the working electrode 21a, the counter electrode 21b, and the reference electrode 21c) are held at stable positions in the wells 8 of the culture container 7, and are immersed in the medium inside the wells 8, which allows the culture state to be properly detected.

Since the bent portions 17 of the sensors 16 are supported from above and below by the support portions 31 and the pressing portions 32, the sensors 16, which are bent at substantially uniform angles with respect to the substrate 13, can be inserted into the wells 8. This allows the sensors 16 to be accurately disposed in the vicinity of the inner peripheral surfaces 8a of the wells 8 (discussed below).

Also, since the radius of the arc portions of the bent portions 17 of the sensors 16 is defined by the bottom cover 14 and the top cover 15, and excessive stress is not exerted on the bent portions 17, disconnection due to cracking can be prevented.

As to the method for bending the bent portion 17, either the top cover 15 or the bottom cover 14 may be bent while attached to the substrate 13. Also, heat may be applied to the bent portions 17 during bending. In this case, the top cover 15 or the bottom cover 14 will be unnecessary.

In this embodiment, as described above, the sensors 16 are formed by cutting out parts of the substrate 13, leaving the bent portions 17, and bending downward with respect to the substrate 13. This eliminates the need for a component for fixing the sensors 16 to the substrate 13, and allows the sensor unit 9 to be more compact.

Also, as the configuration of the sensors 16, since the sensors 16 and the wiring portion on the substrate 13 can be formed integrally, there is no need for connectors between the sensors 16 and the wiring 19, and this also allows the sensor unit 9 to be more compact.

Also, the wiring 19 of the substrate 13 is collected as a wiring pattern on the substrate 13 and bundled into the connection portions 20a and 20b. Since the connection portions 20a and 20b are connected to the connector of the control unit 12, there is no need to connect the sensor unit 9 and the control unit 12 with wiring such as lead wires. This allows the cell culture analysis device 3 itself to be more compact.

As described above, in this embodiment, as shown in FIG. 5, even when many wells 8 are used, their culture states can be simultaneously detected by the sensor unit 9 including a plurality of the small sensors 16.

### Sensors 16

The sensors 16 in this embodiment are formed by cutting out parts of the substrate 13 in an approximate L shape, so the sensors are substantially L-shaped as shown in FIG. 9.

FIG. 10 is a detail cross-sectional view of FIG. 9. In the sensors 16 of this embodiment, as shown in FIG. 10, the upper portion of the vertical side of the approximate L shape is the bent portion 17, and is connected to the substrate 13. That is, the bent portions 17 are provided as connecting portions for connecting the substrate 13 to the sensors 16.

The substantially L-shaped portions 18 of the substrate 13 shown in FIG. 11 are openings from which the substantially L-shaped sensors 16 have been cut out. Also, the substrate 13 is rectangular. The substantially L-shaped sensors 16 are formed by cutting out from the substrate 13 so that the vertical sides of the sensors 16 are at an angle to the two opposing sides of the substrate 13.

Also, as shown in FIG. 11, the wiring 19 on the substrate 13 connected to the bent portions 17 of the sensors 16 is taken out to the outer peripheral portion of the substrate 13 through the space between the vertical side cutout portions 18a of the sensors 16 and the horizontal side cutout portions 18b of the sensors 16, which are adjacent to each other.

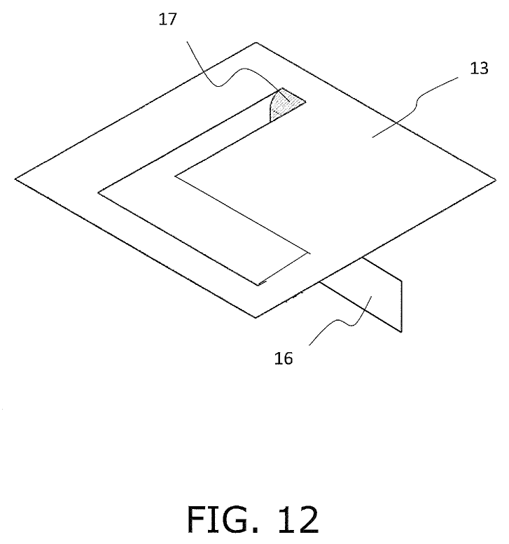

In this embodiment, as shown in FIGS. 9 and 12, the sensors 16 are substantially L-shaped, and the horizontal side portions thereof are immersed in the medium in wells 8 in a state of being held so as to be disposed along the horizontal direction in the wells 8. This allows the state of the cell culture environment in the wells 8 to be detected using the sensors 16 immersed in the wells 8.

Also, detection electrodes (electrode units 21) for detecting the state of the cell culture environment in the wells 8 are formed on the lower lateral side portions of the sensors 16. For example, the sensitivity of the sensors 16 can be improved by increasing the electrode surface area of the electrode units 21 provided as the detection electrodes, as compared to a sensor that is substantially I-shaped.

That is, since the sensors 16 are substantially L-shaped, they are formed by cutting out from the substrate 13 in a state in which the substantially L-shaped vertical sides are at an angle to the two opposite sides of the substrate 13 with respect to the rectangular substrate 13.

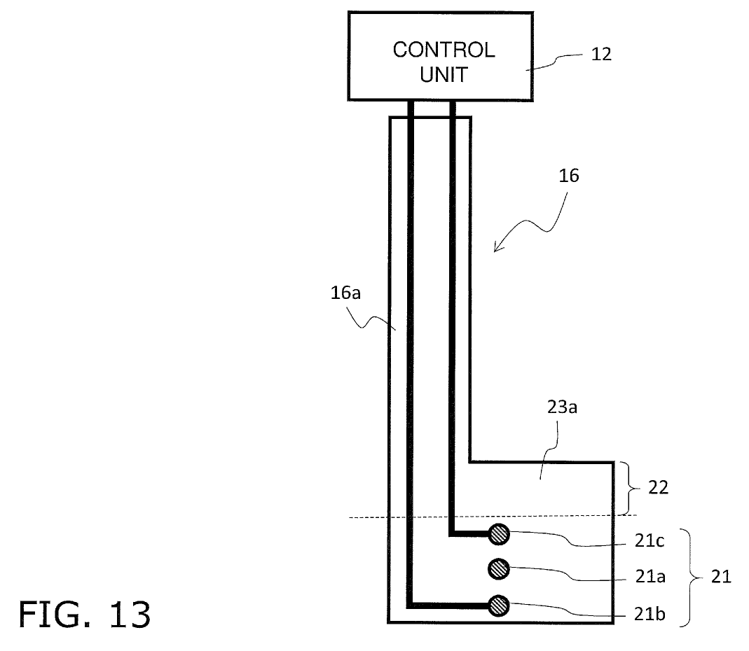

This allows the vertical side portions (the vertically oriented portion in FIG. 13) to have sufficient length. This makes it possible to adjust how far the detection electrode formed on the lateral side portion (the laterally oriented portion in FIG. 13) of a sensor 16 is immersed into the medium in the well 8.

As shown in FIG. 13, a working electrode 21a, a counter electrode 21b, and a reference electrode 21c are provided as the electrode unit 21 on the lateral side portion of the substantially L-shaped sensor 16.

Also, a silver layer (a silver layer and/or a silver chloride layer) is provided on the surface of the reference electrode 21c. A reagent layer formed from an enzyme, a mediator, or the like is provided to the surface of the working electrode 21a. These electrode units 21 are covered with a protective film.

A sensor 16 analyzes the cell culture environment of the medium when the working electrode 21a, the counter electrode 21b, and the reference electrode 21c is immersed in the liquid sample of the medium in the well 8 so that the concentration of a specific component of the medium is electrochemically detected.

For example, when detecting the concentration of the glucose component in the medium, the reagent layer immobilized on the surface of the working electrode 21a contains an enzyme (such as GOx) and a redox mediator.

The principle by which glucose is detected here is that glucose that has permeated from the medium through the protective film is oxidized by an enzymatic reaction with an enzyme (such as GOx) in the reagent layer and becomes gluconolactone, and at the same time, the redox mediator in the reagent layer is reduced into a reductant. The glucose concentration in the medium can be measured by measuring the electrons generated when the reductant returns to an oxidant, as a current value.

The protective film is provided to limit permeation in order to control the permeation rate of glucose in the medium, while preventing permeation to the detection electrode portions of the sensors 16 and the outflow of enzyme and mediator, which are components of the reagent layer immobilized on the working electrode 21a, to the outside of the protective film.

The enzyme and mediator are crosslinked and immobilized on the surface of the working electrode 21a. Therefore, the reagent layer is polymerized to have a larger molecular weight. This prevents glucose from permeating and the enzyme and mediator from flowing out from the protective film (see WO 2019/146788 for further details).

Furthermore, as shown in FIG. 13, each sensor 16 has the above-mentioned electrode unit 21 and the liquid holding portion 22 on one surface (first surface 23a) of the substantially L-shaped main body 16a.

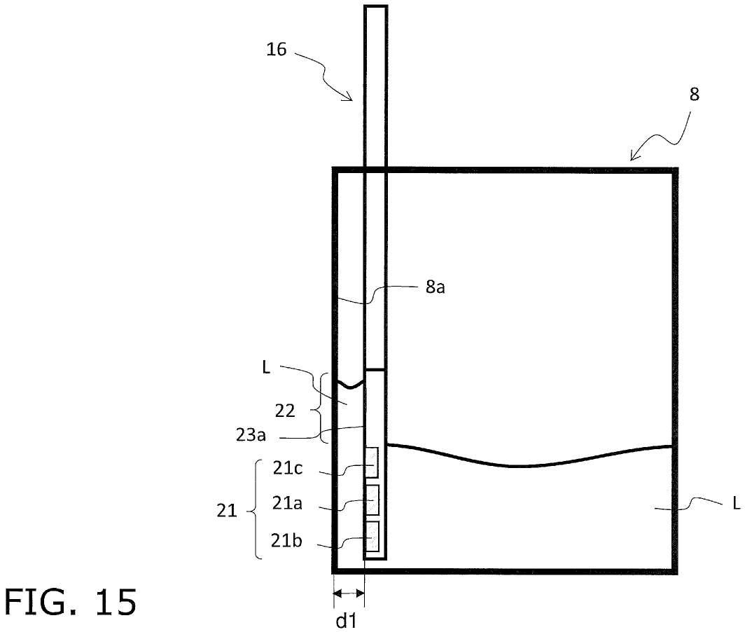

As shown in FIG. 13, the liquid holding portion 22 is provided on the same surface as the surface where the electrode unit 21 is provided, that is, near the electrode unit 21 on the first surface 23a, such as at the upper portion of the electrode unit 21. The liquid holding portion 22 has a width equal to or greater than the width of the portion where the electrode unit 21 is provided. As shown in FIG. 15, the liquid holding portion 22 is formed as a surface disposed at a position higher than the surface level of the medium L held on the first surface 23a side.

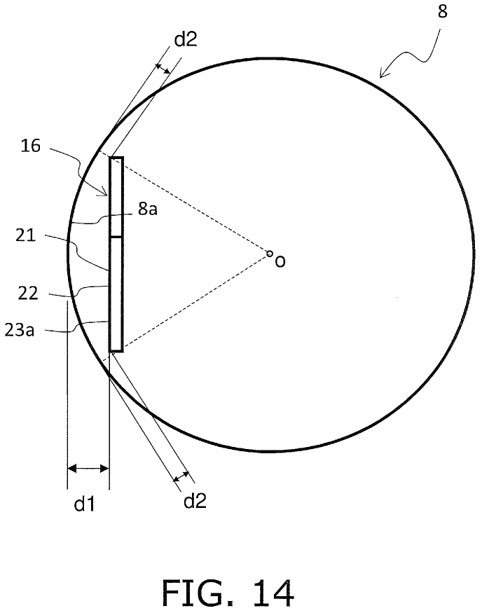

As shown in FIG. 14, each sensor 16 is disposed at a position near the inner peripheral surface (inner wall surface) 8a of the well 8 that is substantially circular in plan view, when a specific voltage is applied to the electrode unit 21 to measure the medium.

At this point, as shown in FIG. 14, the first surface 23a of the sensor 16 is disposed at a position on a chord centered on the center O of the well 8 that is substantially circular in top view.

More specifically, the first surface 23a of the sensor 16 is disposed such that its distance d1 from the inner peripheral surface 8a of the well 8 is 1.0 to 2.0 mm, for example. Also, the first surface 23a is disposed so that the distance d2 from the ends of the sensor 16 to the inner peripheral surface 8a of the well 8 is 1.0 mm, for example.

Consequently, even if the surface level near the center of the well 8 drops due to the meniscus effect that occurs between the medium (liquid sample) contained in the well 8 and the inner peripheral surface 8a of the well 8, because the main body 16a of the sensor 16 is disposed at a position close to the inner peripheral surface 8a of the well 8, all the electrodes included in the electrode unit 21 can be immersed in the medium L.

Here, positioning for disposing a plurality of sensors 16 at positions close to the inner peripheral surfaces 8a of a plurality of wells 8 can be performed by placing the legs 10 of the sensor unit 9 in the positioning holes 11 of the culture container installation unit 6, and placing the culture container 7 in the recess 6a of the culture container installation unit 6.

That is, the positioning of the sensors 16 of the sensor unit 9 with respect to the wells 8 of the culture container 7 is performed by using the recess 6a and the positioning holes 11 formed in the culture container installation unit 6.

As described above, the sensors 16, which are disposed so as to project from the lower surface side of the sensor unit 9, are then accurately disposed at substantially uniform angles with respect to the lower surface of the sensor unit 9 by means of the support portions 31 and the pressing portions 32.

Consequently, the sensor unit 9 (sensors 16) can be positioned with respect to the culture container 7 (wells 8) by positioning the culture container 7 and the sensor unit 9 with respect to the culture container installation unit 6. This allows the sensors 16 to be accurately installed at specific positions near the inner peripheral surfaces 8a of the wells 8.

Therefore, the sensors 16 can be accurately disposed at positions near the inner peripheral surfaces 8a of the wells 8.

Also, the width of the first surface 23a of the main body 16a of a sensor 16 is less than the diameter of the circle of the well 8 that is substantially circular in top view.

Consequently, the sensors 16 can be disposed in a state in which the first surfaces 23a are brought close to the inner peripheral surfaces 8a of the wells 8.

As shown in FIG. 15, the liquid holding portion 22 provided on the first surface 23a side of the main body 16a of a sensor 16 holds the medium (liquid sample) L up to a position that is higher than on the second surface 23b side.

That is, because the first surface 23a of the main body 16a of a sensor 16 is disposed near the inner peripheral surface 8a of the well 8, the liquid holding portion 22 holds the medium up to a position that is higher than on the second surface 23b side by means of the surface tension generated in the medium (liquid sample).

Consequently, even if the surface level near the center of a well 8 drops due to the meniscus effect that occurs between the medium (liquid sample) contained in the well 8 and the inner peripheral surface 8a of the well 8, the surface level will be pushed up on the first surface 23a side where the electrode unit 21 is provided, allowing all of the electrodes contained in the electrode unit 21 to be immersed in the medium L.

As described above, the sensor 16 of this embodiment is disposed at a position that is offset from the center O of the well 8 that is substantially circular in top view so that the electrode unit 21 will be sufficiently immersed in the medium.

That is, the sensor 16 is disposed away from the vicinity of the center of the well 8 where the surface level drops due to the meniscus effect that occurs between the medium (liquid sample) contained in the well 8 and the inner peripheral surface 8a of the well 8.

Consequently, even when the surface level of the medium is lower than that near the inner peripheral surface 8a due to the meniscus effect, since the sensor 16 is disposed near the inner peripheral surface 8a where the surface level is high, all of the electrodes included in the electrode unit 21 can be immersed in the medium L.

### Embodiment 2

The sensor according to another embodiment of the present invention, as well as a measurement device, a sensor unit, a cell culture analysis device, and a liquid sample measuring method comprising this sensor, will be now described with reference to FIGS. 1, 2, and 19 to 36.

### Overview of Cell Culture Device 1

As shown in FIGS. 1 and 2, the cell culture device 1 comprises the culture chamber 2 and the cell culture analysis device 3.

The cell culture analysis device 3 is disposed inside the culture chamber 2 of the cell culture device 1. Although not shown in FIGS. 1 and 2, a door is attached to the front of the culture chamber 2 so as to allow the door to be opened and closed. In the culture chamber 2, cell culture is performed, and the state of the cell culture environment is detected using the sensors 1016 of the cell culture analysis device 3 (discussed below).

### Cell Culture Analysis Device 3

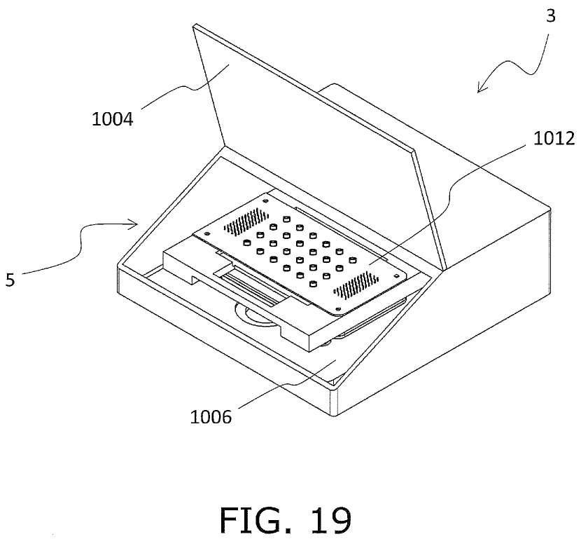

As shown in FIG. 19, the cell culture analysis device 3 comprises a door 1004, the main body case 5, a culture container installation unit 1006, and display units 1038.

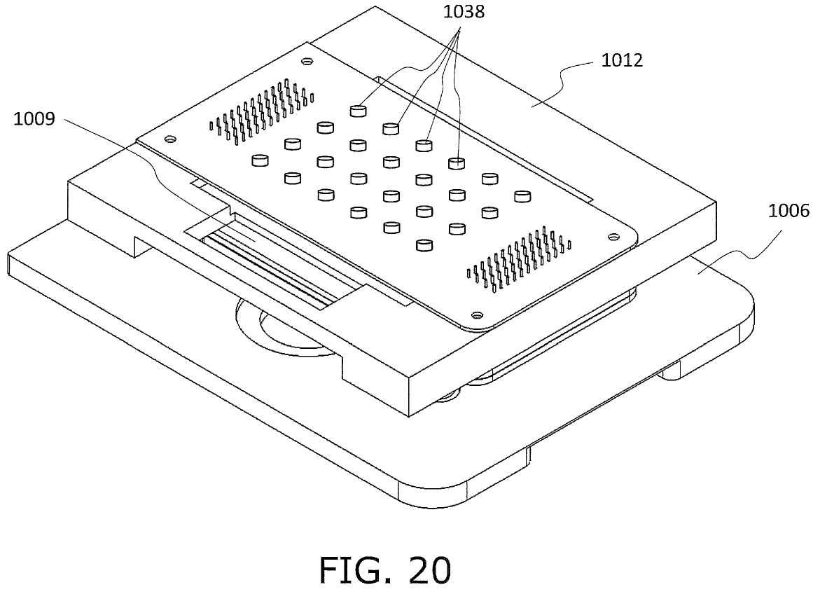

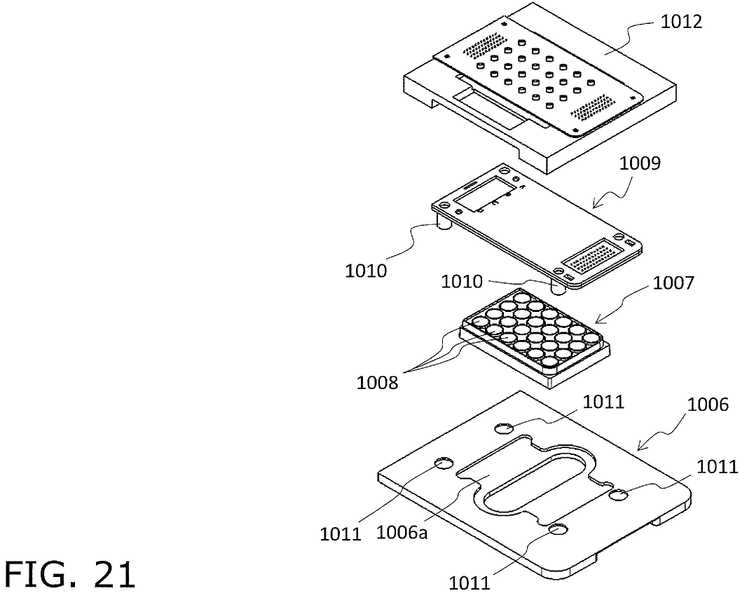

As shown in FIG. 19, the culture container installation unit 1006 is disposed within the main body case 5, which is provided with the door 1004 on its front side. As shown in FIGS. 20 and 21, the culture container 1007 and a sensor unit 1009 are installed on the culture container installation unit 1006.

The display units 1038 are disposed at positions corresponding to the sensors 1016 immersed in the medium in the 24 wells 1008 included in the culture container 1007. The display units 1038 have a one-to-one correspondence with the sensors 1016, and when the immersion state of the measurement-use electrode units 1021 (discussed below) is detected, these display units 1038 are controlled by a control unit 1034 to emit light of a specific color (such as red).

Consequently, the user can recognize on the display unit 1038, from the outside of the cell culture analysis device 3, that the measurement-use electrode unit 1021 is in an unimmersed state when, for example, light of a specific color is not displayed, or when the display is blinking, or when light of a different color is displayed.

As shown in FIG. 21, the culture container 1007 has 24 wells (containers) 1008 (4 up × 6 across), for example. Each of the wells 1008 contains a liquid medium (liquid sample) to be analyzed using the sensor unit 1009.

The wells 1008 are substantially cylindrical containers having a diameter of 15.1 mm, into which a sensor 1016 having a width of about 7.0 mm is inserted, for example. The medium (liquid sample) that is put in each well 1008 has a volume of 0.5 to 1.0 mL, for example.

Also, the culture container 1007 is installed in a state of being positioned in a substantially rectangular recess 1006a formed in the culture container installation unit 1006. The recess 1006a is a recessed portion formed to match the outer shape of the culture container 1007, has substantially the same outer shape as the culture container 1007, and holds the culture container 1007 so as not to move in the plane direction.

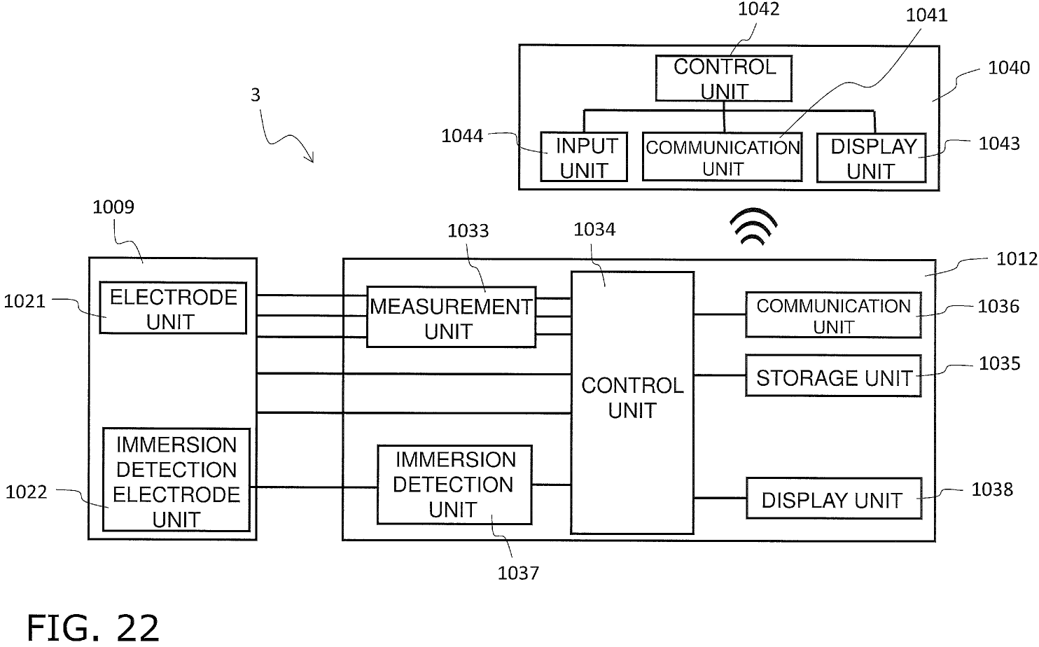

FIG. 22 shows the control blocks of the cell culture analysis device 3.

That is, as shown in FIG. 22, the cell culture analysis device 3 comprises a control unit 1012 including a measurement unit 1033, a control unit 1034, and an immersion detection unit 1037 that are connected to the sensor unit 1009, and a storage unit 1035, a communication unit 1036, and a display unit 1038.

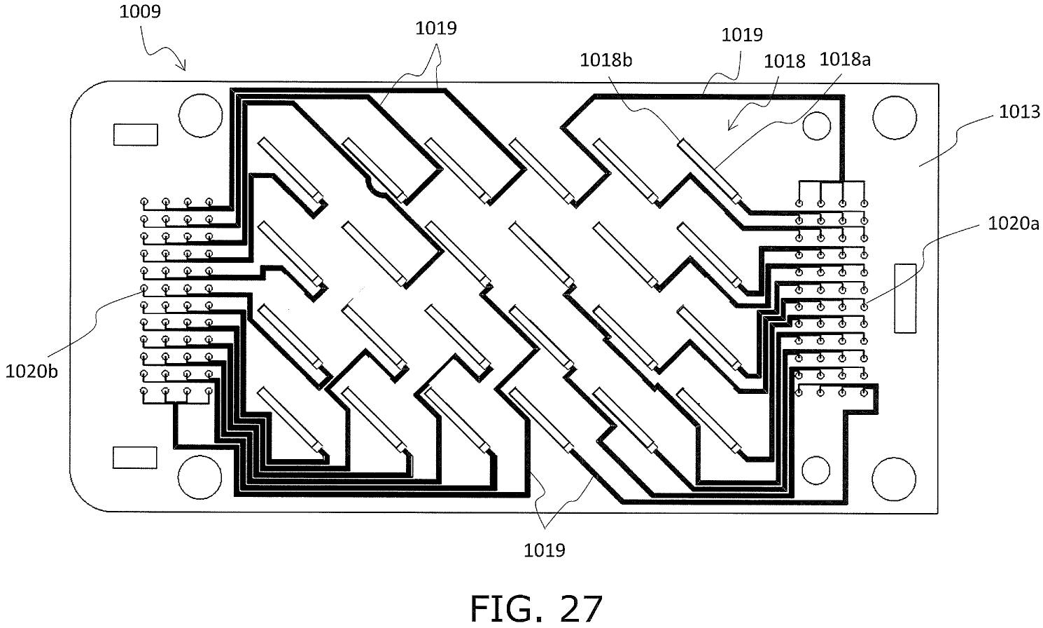

The control unit 1012 applies voltage to the electrode unit (measurement electrode unit) 1021 of each sensor 1016 included in the sensor unit 1009 via connection portions 1020a and 1020b (see FIG. 27). The control unit 1012 transmits information about the cell culture environment of the medium (liquid sample) contained in the wells 1008 to a data processing device (such as a personal computer) external to the cell culture device 1.

Information about the cell culture environment in the wells 1008 detected by the sensors 1016 is transmitted to the control unit 1034 via the measurement unit 1033 provided in the control unit 1012, and is stored in the storage unit 1035. As shown in FIG. 22, information about the cell culture environment stored in the storage unit 1035 is transmitted to the communication unit 1041 of an external device 1040 (such as a personal computer) via the communication unit 1036.

The external device 1040 comprises a communication unit 1041, a control unit 1042, a display unit 1043, and an input unit 1044 (such as a mouse or a keyboard). In the external device 1040, the control unit 1042 controls the display unit 1043 so that the display unit 1043 displays the detected data.

### Sensor Unit 1009

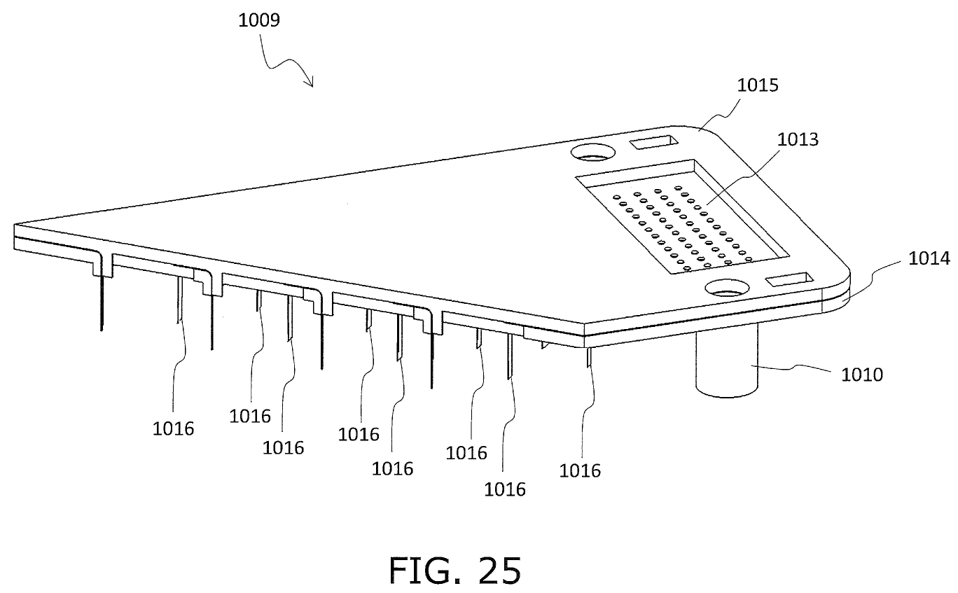

As shown in FIG. 21, the sensor unit 1009, including a plurality of sensors 1016 (see FIG. 25) for analyzing the state of the cell culture environment of the medium (liquid sample) contained in the well 1008, is disposed on the culture container 1007.

As shown in FIG. 21, the sensor unit 1009 has four legs 1010 provided on the lower surface side thereof, and these legs are inserted into positioning holes 1011 provided in the culture container installation unit 1006. As a result, the sensor unit 1009 is installed at a specific position on the culture container installation unit 1006 in a state of being separated from the culture container 1007 by a specific distance.

That is, the legs 1010 for ensuring a space for accommodating the plurality of wells 1008 included in the culture container 7 are provided on the lower surface side of the sensor unit 1009, over the culture container installation unit 1006. The sensor unit 1009 is disposed on the culture container installation unit 1006 by means of the legs 1010.

As described above, the legs 1010 support the sensor unit 1009 on the culture container installation unit 1006 with a specific gap in between in order to ensure enough space for the culture container 7 over the culture container installation unit 6.

Here, the support portions that support the sensor unit 1009 from below are not limited to legs that are provided to the sensor unit 1009. For example, these may be any supports that support the sensor unit 1009 from below with respect to the culture container installation unit 6100.

Also, as shown in FIG. 21, the above-mentioned control unit 1012 is disposed on the sensor unit 1009.

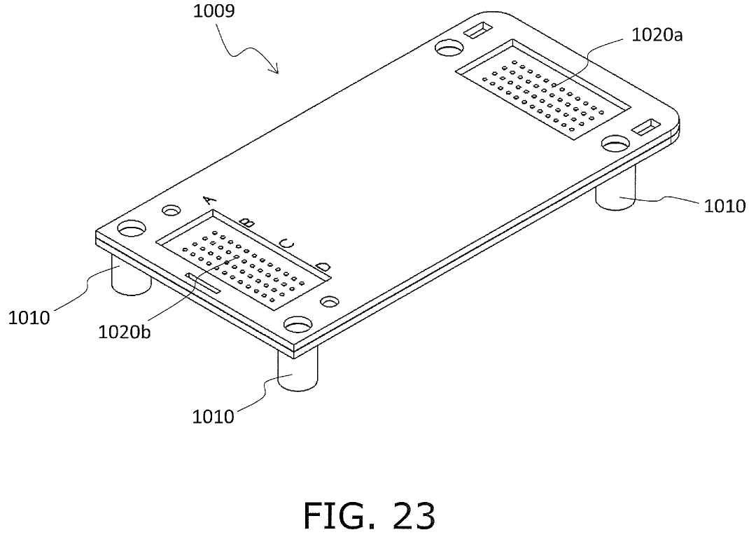

As shown in FIGS. 23 to 25, the sensor unit 1009 comprises, for example, a substrate 1013 molded from PET (polyethylene terephthalate; a resin material), a bottom cover 1014 disposed below the substrate 1013, and a top cover 1015 disposed above the substrate 1013. The substrate 1013 is sandwiched from above and below by the bottom cover 1014 and the top cover 1015.

As shown in FIG. 25, the substrate 1013 is provided with a plurality of sensors 1016. More specifically, the sensors 1016 are formed by cutting out portions of the substrate 1013, leaving on the substrate 1013 bent portions 1017 produced by the downward bending of the connection portions between the sensors 1016 and the substrate 1013.

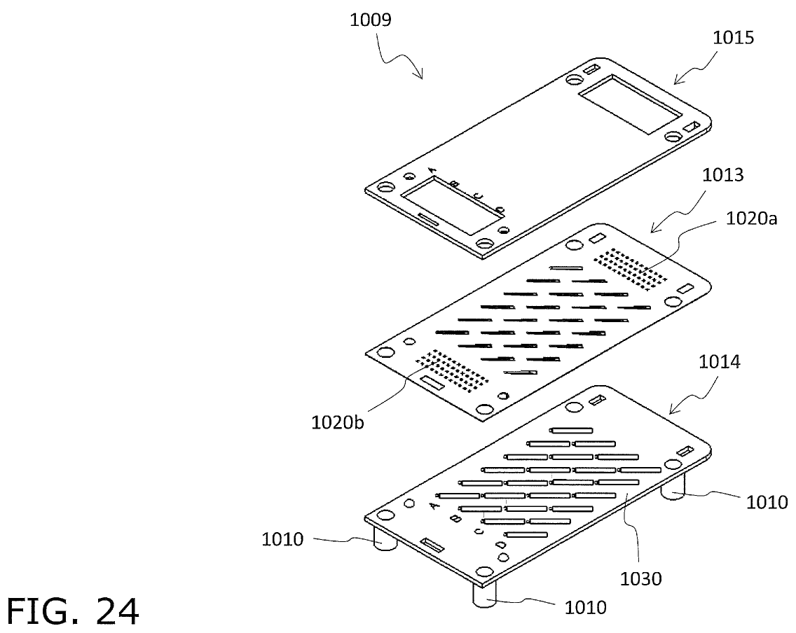

As shown in FIG. 25, the substrate 1013 to which the sensors 1016 are connected is sandwiched between the top cover 1015 and the bottom cover 1014 from above and below. As shown in FIG. 24, the bottom cover 1014 is provided with a plurality of through-holes 1030. Consequently, the lateral side portions of the sensors 1016 (the portions where working electrodes 1021a, counter electrodes 1021b, and reference electrodes 1021c are located) pass through the through-holes 1030 and are disposed below the bottom cover 1014.

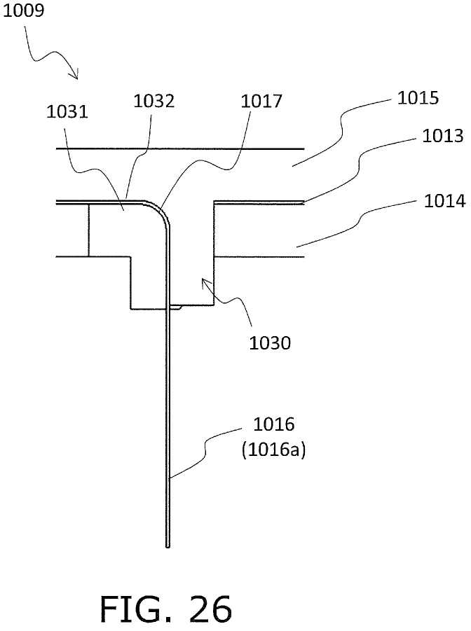

In this embodiment, as shown in FIG. 26, a support portion 1031 that supports the lower side of the bent portion 1017 of each sensor 1016 is provided at the opening edge of each through-hole 1030 in the bottom cover 1014. A pressing portion 1032 that pushes the upper side of the bent portion 1017 of the sensor 1016 downward is provided at the portion of the top cover 1015 that is opposite the support portion 1031.

These support portions 31 have a curved upper surface shape. Also, the pressing portions 32 have a curved lower surface shape.

Therefore, as shown in FIGS. 25 and 26, when the substrate 1013 is sandwiched between the top cover 1015 and the bottom cover 1014 from above and below, the bent portions 1017 of the sensors 1016 are sandwiched from above and below by the support portions 1031 and the pressing portions 1032.

Consequently, the lateral side portions of the substantially L-shaped sensors 1016 (the portion provided with the working electrode 1021a and the counter electrode 1021b) can be stably maintained in a state of being disposed along the substantially horizontal direction.

Thus, the lateral side portions of the sensors 1016 (the portion provided with the working electrode 1021a and the counter electrode 1021b) are held at stable positions in the wells 1008 of the culture container 1007, and are immersed in the medium inside the wells 1008, which allows the culture state to be properly detected.

Since the bent portions 1017 of the sensors 1016 are supported from above and below by the support portions 1031 and the pressing portions 1032, the sensors 1016, which are bent at substantially uniform angles with respect to the substrate 1013, can be inserted into the wells 1008. This allows the sensors 1016 to be accurately disposed at specific positions within the wells 1008 (discussed below).

Also, since the radius of the arc portions of the bent portions 1017 of the sensors 1016 is defined by the bottom cover 1014 and the top cover 1015, and excessive stress is not exerted on the bent portions 1017, disconnection due to cracking can be prevented.

As to the method for bending the bent portion 1017, either the top cover 1015 or the bottom cover 1014 may be bent while attached to the substrate 1013. Also, heat may be applied to the bent portions 1017 during bending. In this case, the top cover 1015 or the bottom cover 1014 will be unnecessary.

In this embodiment, as described above, the sensors 1016 are formed by cutting out parts of the substrate 1013, leaving the bent portions 1017, and bending downward with respect to the substrate 1013. This eliminates the need for a component for fixing the sensors 1016 to the substrate 1013, and allows the sensor unit 1009 to be more compact.

Also, as the configuration of the sensors 1016, since the sensors 1016 and the wiring portion on the substrate 1013 can be formed integrally, there is no need for connectors between the sensors 1016 and the wiring 1019, and this also allows the sensor unit 1009 to be more compact.

Also, the wiring 1019 of the substrate 1013 is collected as a wiring pattern on the substrate 1013 and bundled into the connection portions 1020a and 1020b. Since the connection portions 1020a and 1020b are connected to the connector of the control unit 1012, there is no need to connect the sensor unit 1009 and the control unit 1012 with wiring such as lead wires. This allows the cell culture analysis device 3 itself to be more compact.

As described above, in this embodiment, as shown in FIG. 21, even when a culture container 1007 including many wells 1008 is used, the culture states can be simultaneously detected by the sensor unit 1009 including a plurality of the small sensors 1016.

### Sensors 1016

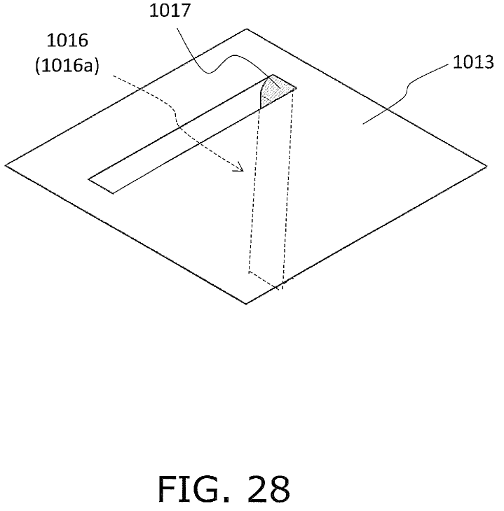

The sensors 1016 in this embodiment are formed by cutting out parts of the substrate 1013 in an approximate L shape, so the sensors are substantially L-shaped as shown in FIGS. 25 and 28.

FIG. 26 is a detail cross-sectional view of FIG. 25. In the sensors 1016 of this embodiment, as shown in FIG. 26, the upper portion of the vertical side of an substantially I-shaped main body 1016a serves as the bent portion 1017, and is connected to the substrate 1013. That is, the bent portions 1017 are provided as connecting portions for connecting the substrate 1013 to the sensors 1016.

The substantially I-shaped portions 1018 of the substrate 13 shown in FIG. 27 are openings from which the substantially I-shaped sensors 1016 have been cut out. Also, the substrate 1013 is rectangular. The substantially I-shaped sensors 1016 are formed by cutting out from the substrate 1013 so that the vertical sides of the sensors 1016 are at an angle to the two opposing sides of the substrate 1013.

Also, as shown in FIG. 27, the wiring 1019 on the substrate 1013 connected to the bent portions 1017 of the sensors 1016 is taken out to the outer peripheral portion of the substrate 1013 through the space between the vertical side cutout portions 1018a of the sensors 1016 and the horizontal side cutout portions 1018b of the sensors 1016, which are adjacent to each other.

In this embodiment, as shown in FIGS. 26 and 28, the main body 1016a of the sensor 1016 is substantially I-shaped, and is immersed in the medium in the well 1008 in a state in which the lateral side portions thereof are held so as to be disposed along the horizontal direction within the well 1008. Consequently, the state of the cell culture environment in the plurality of wells 1008 can be detected by using the sensors 1016 immersed in the wells 1008.

Also, below a sensor 1016 is formed an electrode unit 1021 for detecting the state of the cell culture environment in the well 1008 (such as the concentration of the specific component contained in the medium L).

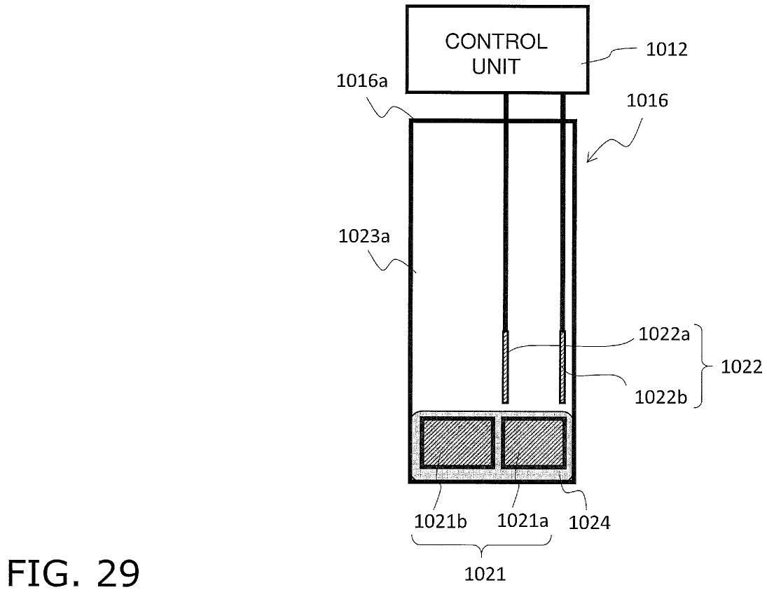

This ensures that the vertical side portion of the sensor 1016 (the portion extended along the vertical direction in FIG. 29) will be sufficiently long, so detection electrode formed in the horizontal side portion of the sensor 1016 (the portion extended along the left and right direction in FIG. 29) can be immersed in the medium in the well 1008.

As shown in FIG. 29, a working electrode 1021a and a counter electrode 1021b are provided as the electrode unit 1021 near the lower end of the substantially I-shaped sensor 1016.

Also, a reagent layer formed from an enzyme, a mediator, etc., is provided on the surfaces of the working electrode 1021a and the counter electrode 1021b. The electrode unit 1021 including the working electrode 1021a and the counter electrode 1021b is covered by a protective film 1024.

The sensor 1016 analyzes the cell culture environment of the medium when the working pole 1021a and the counter pole 1021b are immersed in the medium L in the well 1008, so that the concentration of a specific component of the medium is electrochemically detected.

For example, when detecting the concentration of the glucose component contained in the medium, the reagent layer immobilized on the surface of the working electrode 1021a contains an enzyme (such as GOx) and a redox mediator.

The principle behind this glucose detection is that glucose that permeates from the medium through the protective film 1024 is oxidized by an enzymatic reaction with the enzyme (such as GOx) in the reagent layer to become gluconolactone, and at the same time the redox mediator in the reagent layer is reduced to become a reductant. The glucose concentration in the medium can be measured by measuring, as a current value, the electrons generated when this reductant goes back to being an oxidant.

The protective film 1024 is provided to limit permeation so as to control the permeation rate of glucose in the medium, and to cause glucose to permeate into the detection electrode units of the sensors 1016. Furthermore, the protective film 1024 is provided to prevent the enzyme and the mediator, which are components of the reagent layer immobilized on the working electrode 1021a, from flowing out to the outside of the protective film 1024 (into the medium).

The enzyme and the mediator are cross-linked and immobilized on the surface of working electrode 1021a. Therefore, the reagent layer is polymerized and has a large molecular weight. Consequently, glucose can permeate the reagent layer, but the enzyme and mediator can be prevented from permeating through the protective membrane 1024 and flowing out (see WO 2019/146788 for more details).

Furthermore, as shown in FIG. 29, the sensor 1016 has the above-mentioned electrode unit 1021 and the immersion detection electrode unit 1022 on one surface (first surface 1023a) of the substantially I-shaped main body 1016a.

As shown in FIG. 29, the immersion detection electrode unit 1022 is provided on the same surface as the one on which the measurement-use electrode unit 1021 is provided, that is, directly above the electrode unit 1021 on the first surface 1023a, such as at the upper part of the working electrode 1021a included in the electrode unit 1021. Also, the immersion detection electrode unit 1022 has two electrodes (first electrode 1022a and second electrode 1022b). The first electrode 1022a and the second electrode 1022b are installed above the working electrode 1021a at a spacing that is substantially equal to the width of the working electrode 1021a of the measurement-use electrode unit 1021.

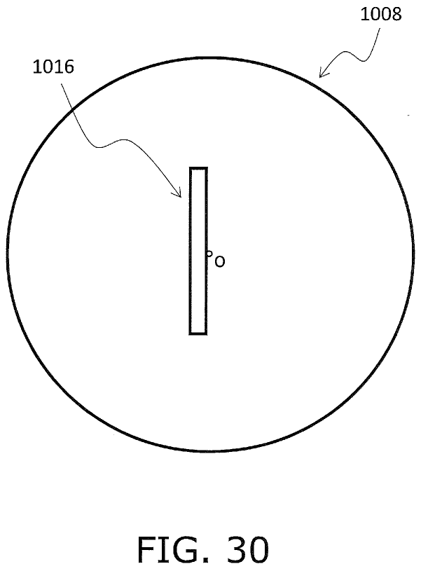

When a specific voltage is applied to the electrode unit 1021 to measure the medium, the sensor 1016 is disposed in the center (near the center O) of the well 1008 that is substantially circular in top view, as shown in FIG. 30.

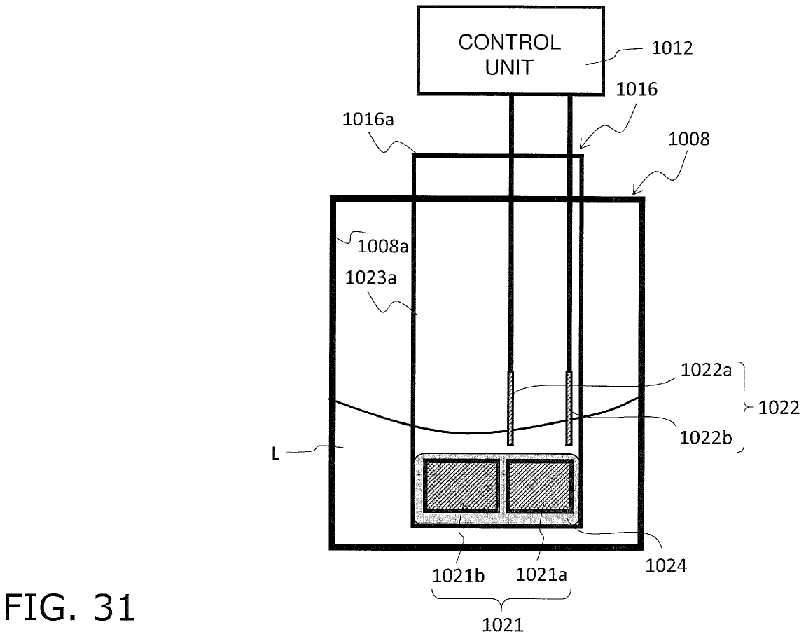

Here, as shown in FIG. 31, in the medium (liquid sample) L contained in the well 1008, the surface level near the center O of the well 1008 drops due to the meniscus effect that occurs between the medium and the inner peripheral surface 1008a of the well 1008. Therefore, with the sensor 1016 in this embodiment, the main body 1016a of the sensor 1016 is disposed near the center O of the well 1008 where the surface level of the medium L is expected to be the lowest, and the immersion state of the electrode unit 1021 in the medium L is thus detected, making it possible to detect whether or not the electrode unit 1021 is immersed far enough in the medium L.

Here, positioning for disposing the plurality of sensors 1016 near the center O of the plurality of wells 1008 is accomplished by placing the above-mentioned legs 1010 of the sensor unit 1009 in the positioning holes 1011 of the culture container installation unit 1006, and placing the culture container 1007 in the recess 1006a of the culture container installation unit 1006.

That is, the positioning of the sensors 1016 of the sensor unit 1009 with respect to the wells 1008 of the culture container 1007 is performed by using the recess 1006a and the positioning holes 1011 formed in the culture container installation unit 1006.

As described above, the sensors 1016, which are disposed so as to project from the lower surface side of the sensor unit 1009, are accurately disposed at a substantially uniform angle with respect to the lower surface of the sensor unit 1009, by means of the support portions 1031 and the pressing portions 1032.

Consequently, the sensor unit 1009 (the sensors 1016) can be positioned with respect to the culture container 1007 (the wells 1008) by positioning the culture container 1007 and the sensor unit 1009. This allows the sensors 1016 to be accurately installed at a position near the center O of each well 1008.

Therefore, the sensors 1016 can be accurately disposed in the wells 1008.

Also, with the sensor 1016 in this embodiment, the immersion detection electrode unit 1022 is disposed on the working electrode 1021a included in the measurement-use electrode unit 1021.

Here, the change in the immersion state of the working electrode 1021a in the medium L tends to affect the current value measured by applying a voltage to the electrode unit 1021, more than with the other electrodes (the counter electrode 1021b, etc.). Therefore, with the sensor 16 in this embodiment, the immersion detection electrode unit 1022 is disposed on the working electrode 1021a in order to reliably detect the immersion state of the working electrode 1021a, which tends to affect the measurement due to a change in the immersion state.

Consequently, the immersion state of the working electrode 1021a can be reliably detected by applying a voltage to the first electrode 1022a and the second electrode 1022b of the immersion detection electrode unit 1022.

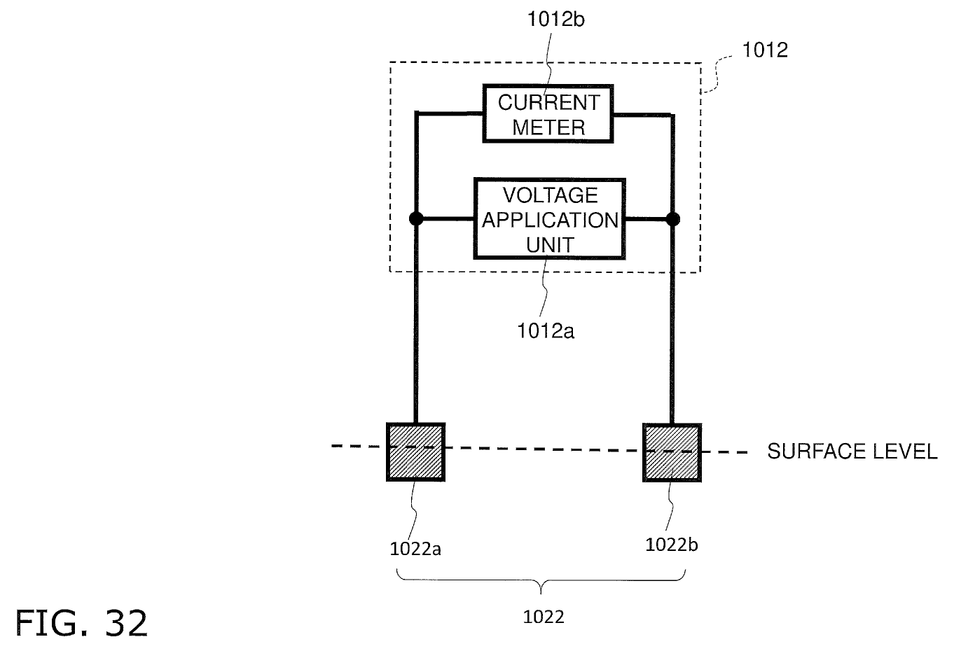

As shown in FIG. 32, the control unit 1012 functions as a measurement device comprising a voltage application unit 1012a that applies a specific voltage (second voltage) between the first electrode 1022a and the second electrode 1022b of the sensor 1016, and a current meter 1012b that measures the current flowing between the first electrode 1022a and the second electrode 1022b. That is, when detecting the immersion state of the electrode unit 1021, a specific voltage (second voltage) is applied from the voltage application unit 1012a to the immersion detection electrode unit 1022 (first electrode 1022a and second electrode 1022b), and the current flowing between the first electrode 1022a and the second electrode 1022b is measured by the current meter 1012b.

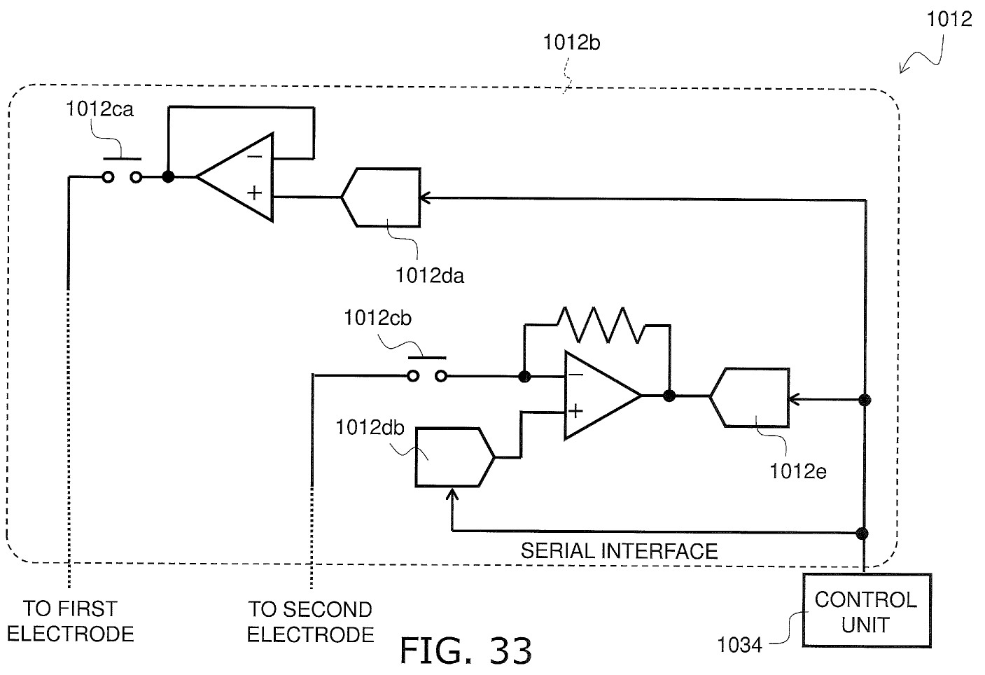

More precisely, as shown in FIG. 33, the control unit 1034 applies 1.0 V, as a specific voltage (second voltage) for immersion detection, to each of the D/A (digital/analog) converters 1012db and 1012da included in the circuit shown in FIG. 33. Then the switch 1012ca is then moved from OFF to ON, after which the switch 1012 cc is moved from OFF to ON to set the current value flowing between the first electrode 1022a and the second electrode 1022b in the current meter 1012b.

The current meter 1012b includes a resistor, an operational amplifier, and an A/D (analog/digital) converter 1012e, and detects a minute current flowing between the first electrode 1022a and the second electrode 1022b.

The current value measured by the current meter 1012b increases proportionally with the surface area in which the first electrode 1022a and the second electrode 1022b are immersed in the medium L.

Consequently, when the current value detected by the current meter 1012b is at or above a specific threshold value, for example, it can be detected that the immersion detection electrode unit 1022 is immersed in the medium L, that is, that the measurement-use electrode unit 1021 disposed directly under the immersion detection electrode unit 1022 is immersed in the medium L.

As shown in FIG. 31, the fact that the detected current value changes according to the change in the immersed surface area of the first electrode 1022a and the second electrode 1022b may be utilized to determine the surface level of the medium L according to the magnitude of the current value detected by the current meter 1012b when electrodes that are longer in the immersion depth direction (substantially vertical direction) (first electrode 1022a and second electrode 1022b) are used.

Also, as described above, the two electrodes (first electrode 1022a and second electrode 1022b) constituting the immersion detection electrode unit 1022 are installed so as to match the width of the working electrode 1021a. Therefore, at least whether or not the working electrode 1021a, which affects the measurement result, is in an immersed state can be detected by applying a specific voltage to the immersion detection electrode unit 1022 and detecting the immersion state thereof.

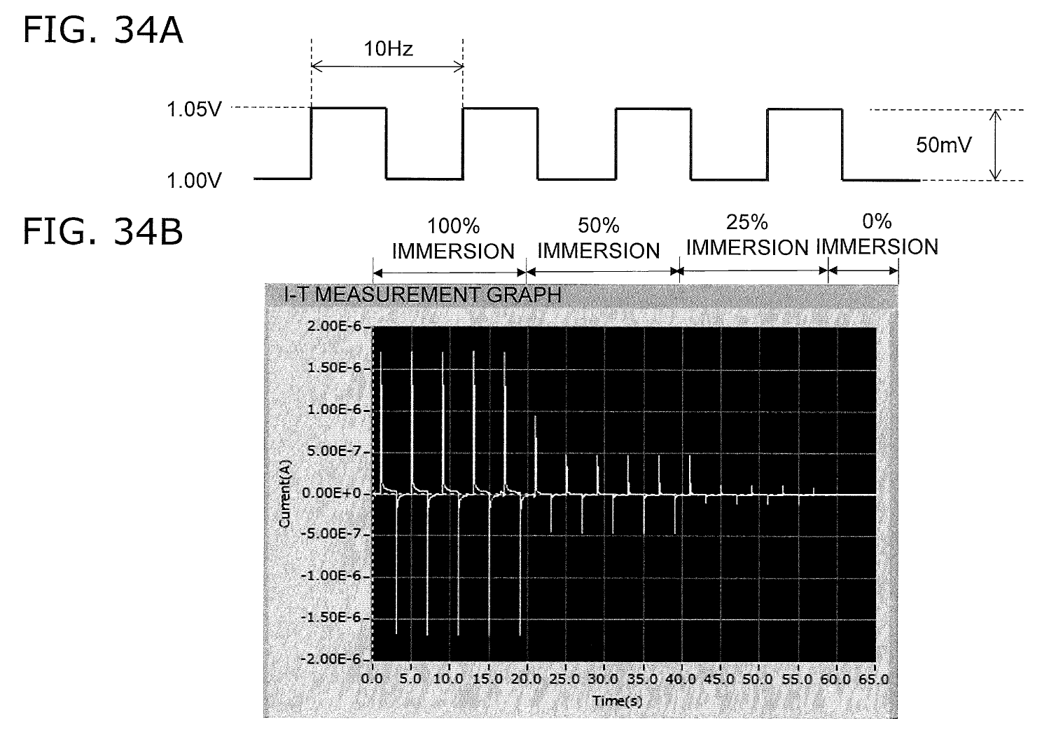

As shown in FIG. 34A, the voltage application unit 1012a applies a square wave (duty: 50%, frequency: 10 Hz, amplitude: 50 mV) voltage to the first electrode 1022a and the second electrode 1022b.

Here, the current value measured in order to detect the immersion state of the measurement-use electrode unit 1021 is converted from current to voltage in a transimpedance circuit on the second electrode 1022b side shown in FIG. 33, and then inputted to the A/D converter 1012e.

At this point, if the immersion depth of the first electrode 1022a and the second electrode 1022b changes in the direction of becoming shallower over time, the current value detected by the current meter 1012b is changes in the direction of becoming smaller, as shown in FIG. 34B.

More specifically, if the immersion state is varied such that from 0 to 20 seconds on the horizontal axis shown in FIG. 34B, the immersion depth is at the maximum (the entire surface area of the first electrode 1022a and the second electrode 1022b is immersed), from 20 to 40 seconds the immersion is 50%, from 40 to 60 seconds the immersion depth is 25%, and from 60 seconds onward the immersion depth is 0%, the detected current value also changes accordingly in the direction of becoming smaller.

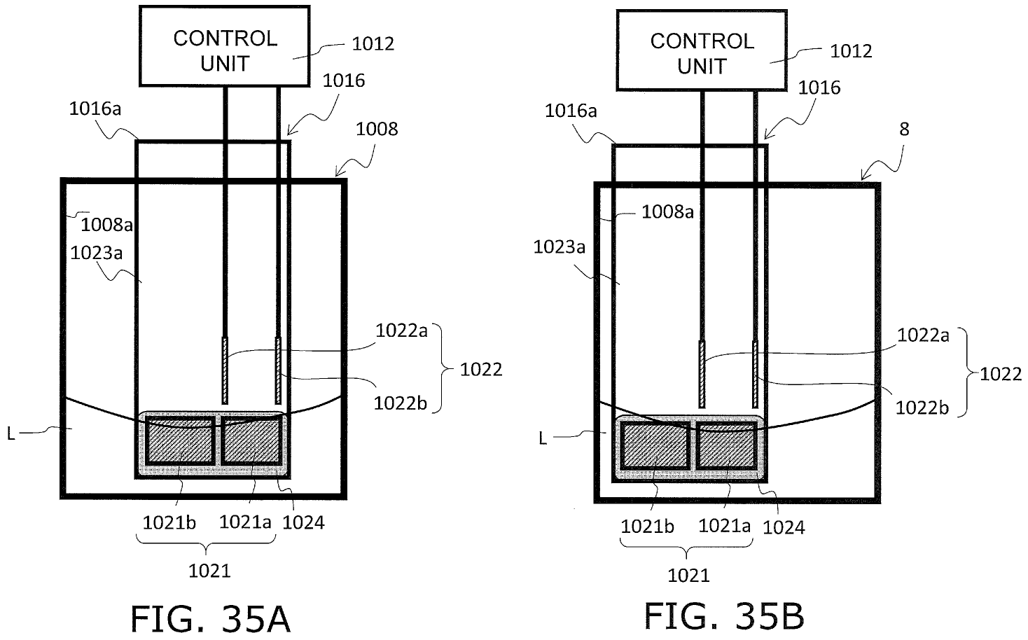

Consequently, even when the surface level of the medium L has dropped as shown in FIG. 35A, or when the sensor 1016 is offset to be closer to the inner peripheral surface 1008a of the well 1008 the surface level of the medium L has dropped as shown in FIG. 35B, for example, it can still be detected that the first electrode 1022a and the second electrode 1022b of the immersion detection electrode unit 1022 are not immersed.

Consequently, it can be detected that part of the measurement-use electrode part 1021 (such as a part of the working electrode 1021a and a part of the counter electrode 1021b) disposed below the lower end of the immersion detection electrode part 1022 is not immersed, and measures can be taken, such as adding more of the medium L or increasing the immersion depth of the sensor 1016.

As a result, it is possible to prevent measurement from being performed in a state in which part of the measurement-use electrode unit 1021 (such as a part of the working electrode 1021a and a part of the counter electrode 1021b) is not immersed, which would lower measurement accuracy.

Also, in this embodiment, as described above, the voltage application unit 1012a applies the AC wave (square wave) voltage shown in FIG. 34A to the first electrode 1022a and the second electrode 1022b.

Consequently, a larger current value can be obtained as compared to when a DC wave voltage is applied, and the S/N ratio and the detection speed can be improved.

### Method for Measuring Liquid Sample

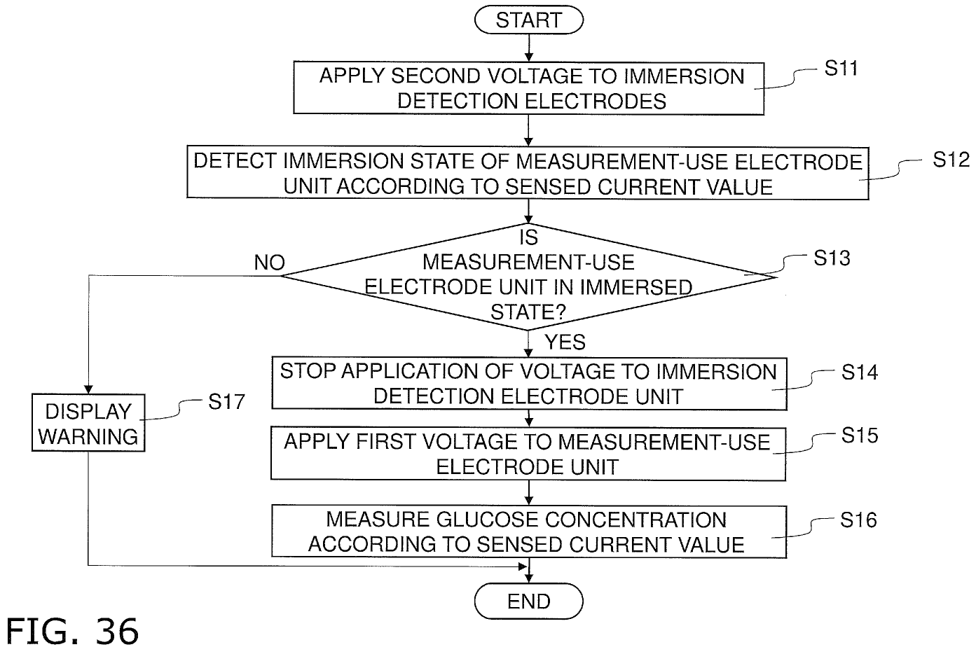

In the measurement method of this embodiment, processing is performed according to the flowchart shown in FIG. 36 in order to measure the concentration of glucose contained in the medium L described above,

In step S11, the voltage application unit 1012a applies a specific voltage (second voltage) to the immersion detection electrode unit 1022 (first electrode 1022a and second electrode 1022b) in order to detect the immersion state of the measurement-use electrode unit 1021.

Next, in step S12, the immersion state of the measurement-use electrode unit 1021 is detected according to the current value sensed by the current meter 1012b.

Next, in step S13, it is determined from the detection result in step S12 whether or not the measurement-use electrode unit 1021 is in an immersed state. If the answer is Yes, the processing proceeds to step S14, and if the answer is No because there is too little medium L or for another such reason, the processing proceeds to step S17.

Next, in step S14, since it was determined in step S13 that the measurement-use electrode unit 1021 is in an immersed state, the voltage application unit 1012a stops applying the second voltage to the detection electrode unit 1022 in order to begin measuring the medium (liquid sample) L.

Next, in step S15, the voltage application unit 1012a applies the measurement-use first voltage to the measurement-use electrode unit 1021.

Next, in step S16, the concentration of a specific component (glucose) contained in the medium L is measured according to the current value detected by the current meter 1012b, and the processing is ended.

On the other hand, if it is determined in step S13 that the measurement-use electrode unit 1021 is not in an immersed state, in step S17 the above-mentioned display unit 1038 is used to display a warning at the position corresponding to the sensor 1016 determined not to be in an immersed state.

Here, the warning display on the display unit 1038 includes, for example, that a light of a specific color to be lit when a normal immersion state is detected is not turned on (stays off), or that the light is flashed, or that light of a different color is turned on.

This makes it easy for the user to recognize at which position the measurement-use electrode unit 1021 of a sensor 1016 placed in a well 1008 is not immersed in the medium L.

As a result, the user can take measures such as adding more medium L to the well 1008 in which the sensor 1016 at the corresponding position is installed, ignoring the measurement result from the sensor 1016 at the corresponding position, and so forth. This makes it possible to improve measurement accuracy by preventing a decrease in measurement accuracy attributable to the immersion state of the measurement-use electrode unit 1021 in measurement using the sensor 1016.

### Embodiment 3

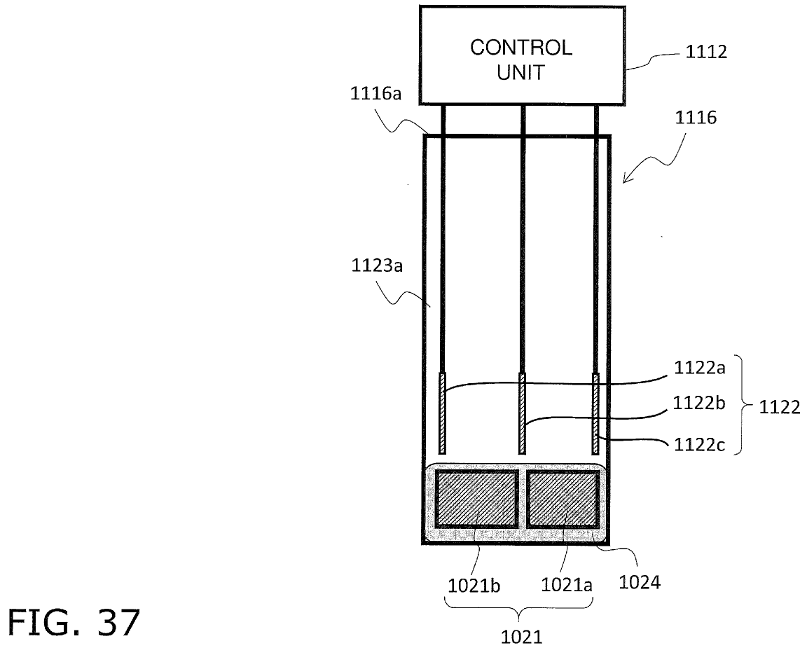

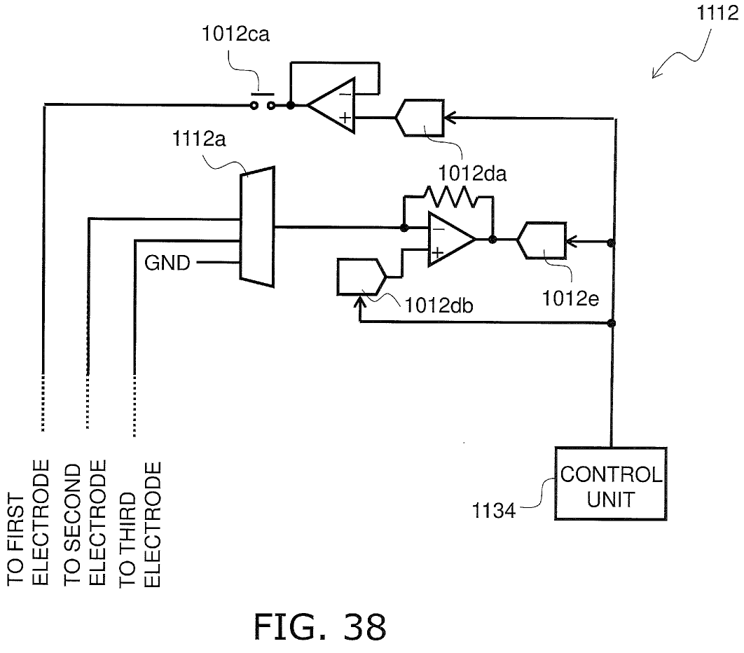

The configuration of a sensor 1116 according to yet another embodiment of the present invention will now be described with reference to FIGS. 37 and 38.

In this embodiment, three electrodes (the first electrode 1122a, second electrode 1122b, and third electrode 1122c) are used as the immersion detection electrode unit 1122 for detecting the immersion state of the measurement-use electrode unit 1021, and differs in this respect from Embodiment 2 above in which two electrodes (the first electrode 22a and second electrode 22b) are used.

That is, as shown in FIG. 37, the sensor 1116 of this embodiment comprises an immersion detection electrode unit 1122 including three electrodes (a first electrode 1122a, a second electrode 1122b, and a third electrode 1122c) directly above the measurement-use electrode unit 1021 provided near the lower end portion of the first surface 1123a of the main body 1116a.

Of the three electrodes 1122a to 1122c, the first electrode 1122a and the third electrode 1122c are disposed near the two ends in the width direction of the main body 1116a of the sensor 1116, directly above the measurement-use electrode unit 1021. The second electrode 1122b is disposed near the approximate center in the width direction of the main body 1116a of the sensor 1116, directly above a position between the working electrode 1021a and the counter electrode 1021b of the measurement-use electrode unit 1021.

As shown in FIG. 38, the control unit 1112 comprises a selection circuit 1112a. When the control unit 1134 selects two electrodes to which voltage is to be applied, the immersion state between the first electrode 1122a and the second electrode 1122b, and the immersion state between the first electrode 1122a and the third electrode 1122c are each detected. This makes it possible to detect the immersion state of the first electrode 1122a, the second electrode 1122b, and the third electrode 1122c.

This makes it possible to accurately detect whether or not the working electrode 1021a and the counter electrode 1021b included in the measurement electrode unit 1021 shown in FIG. 37 are totally and individually immersed.

### Embodiment 4

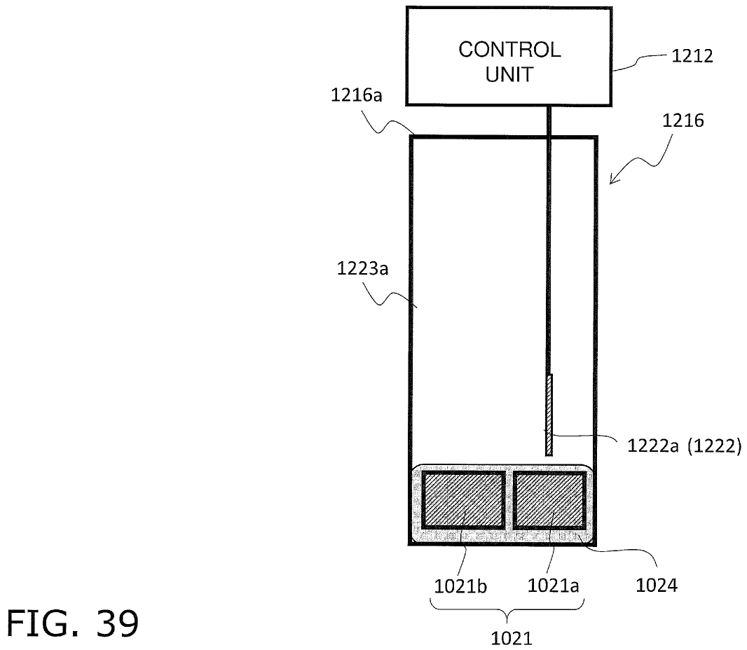

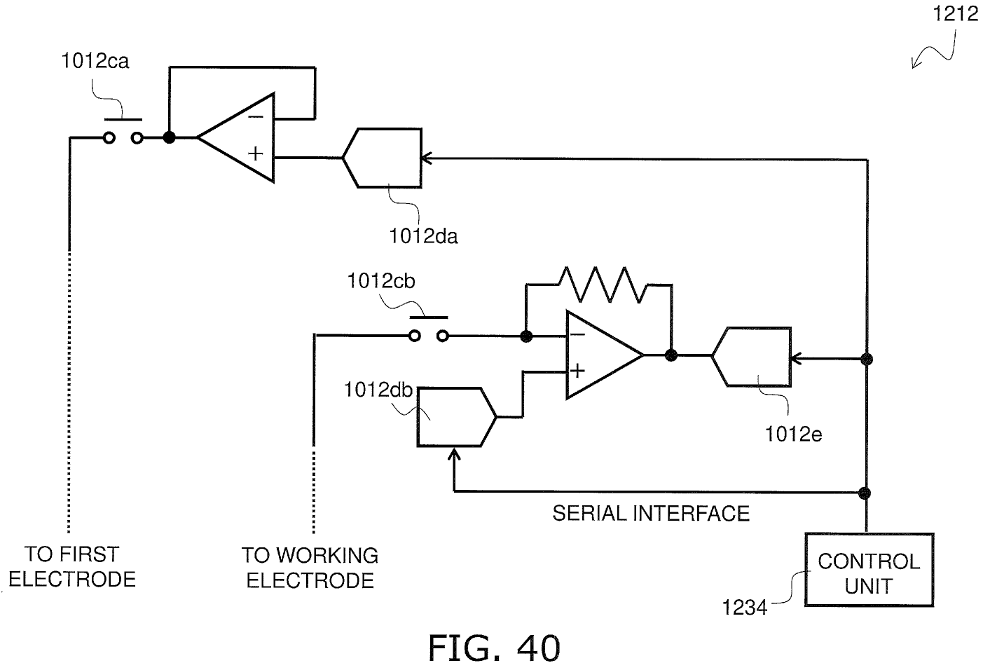

The configuration of a sensor 1216 according to yet another embodiment of the present invention will now be described with reference to FIGS. 39 and 40.

In this embodiment, the immersion detection electrode unit 1222 for detecting the immersion state of the measurement-use electrode unit 1021 is a combination of one electrode (a first electrode 1222a) and the working electrode 1021a of the measurement-use electrode unit 1021, and differs in this respect from Embodiment 2 above in which two electrodes (the first electrode 22a and the second electrode 22b) are used.

That is, as shown in FIG. 39, the sensor 1216 of this embodiment comprises an immersion detection electrode unit 1222 including one electrode (first electrode 1222a), directly above the measurement-se electrode unit 1021 provided near the lower end portion of the first surface 1223a of the main body 1216a.

The first electrode 1222a is disposed near the end in the width direction of the main body 1216a of the sensor 1216 on the side where the working electrode 1021a is disposed, directly above the working electrode 1021a.

As shown in FIG. 40, the control unit 1212 can detect the immersion state between the first electrode 1222a and the working electrode 1021a when a voltage for immersion detection between the first electrode 1222a and the working electrode 1021a is applied by the control unit 1234.

This makes it possible to accurately detect whether or not the working electrode 1021a included in the measurement-use electrode unit 1021 shown in FIG. 39 is immersed.

The configuration of the sensor 1216 can be simplified by using one first electrode 1222a as the immersion detection electrode unit 1222, and using a circuit including the working electrode 1021a of the measurement-use electrode unit 1021 to help with immersion detection.

### Embodiment 5

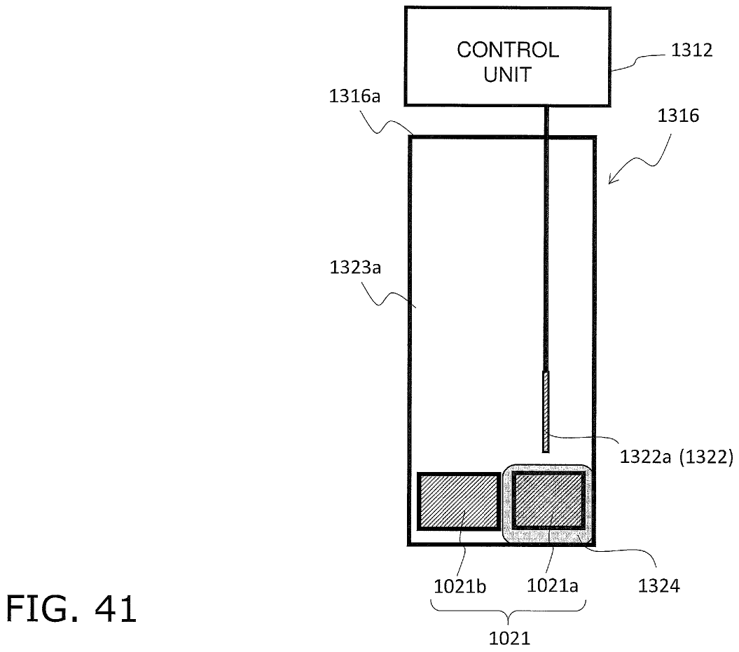

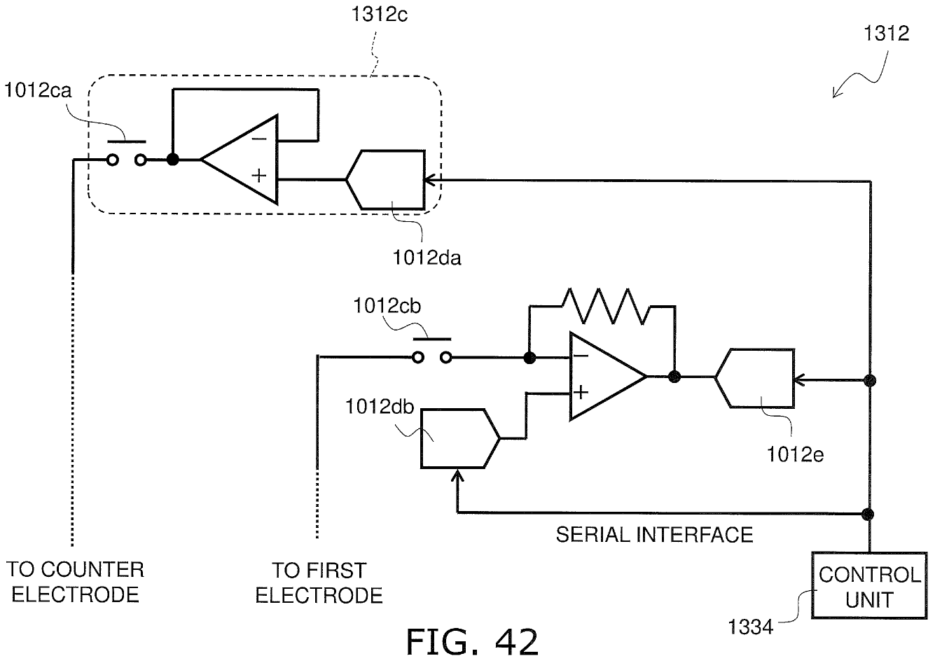

The configuration of a sensor 1316 according to yet another embodiment of the present invention will now be described with reference to FIGS. 41 and 42.

In this embodiment, of the working electrode 1021a and the counter electrode 1021b constituting the measurement-use electrode unit 1021, only the working electrode 1021a is covered with the protective film 1324, and this differs from Embodiment 4 above in which the working electrode 1021a and the counter electrode 1021b are both covered by the protective film 1024. Furthermore, this embodiment differs from Embodiments 2 to 4 above in that in performing immersion detection, voltage is applied between the first electrode 1322a of the immersion detection electrode unit 1322 and the counter electrode 1021b by using the counter electrode side circuit 1312c.

That is, as shown in FIG. 41, the sensor 1316 of this embodiment comprises a protective film 1324 that is provided so as to cover the working electrode 1021a near the lower end portion of the first surface 1323a of the main body 1316a.

The protective film 1324 is disposed so as to cover only the working electrode 1021a provided near the end portion in the width direction of the main body 1316a of the sensor 1316.

As shown in FIG. 42, the control unit 1312 can detect the immersion state of the measurement-use electrode unit 1021 when a voltage for immersion detection is applied between the first electrode 1322a and the counter electrode 1021b by the control unit 1334.

This makes it possible to accurately detect whether or not the measurement electrode unit 1021 shown in FIG. 41 is immersed in the medium L.

The configuration of the sensor 1316 can be simplified by using one first electrode 1322a as the immersion detection electrode unit 1322, and using a circuit including the counter electrode 1021b of the measurement-use electrode unit 1021 to help with immersion detection, and providing the protective film 1324 on only the working electrode 1021a side.

As described above, the protective film 1324 is provided so that the components contained in the reagent layer provided on the working electrode 1021a will not dissolve into the medium L, and is therefore preferably provided so as to cover at least the working electrode 1021a.

On the other hand, since the counter electrode 1021b is not provided with a reagent layer, when voltage is applied between the first electrode 1322a and the counter electrode 1021b, the immersion state of the working electrode 1021a can be detected as soon as the sensor 1316 is immersed in the medium L, without any waiting time for permeating through the protective film. Consequently, the immersion state can be detected more quickly than when voltage for immersion detection is applied between the first electrode 1322a and the working electrode 1021a.

### Embodiment 6

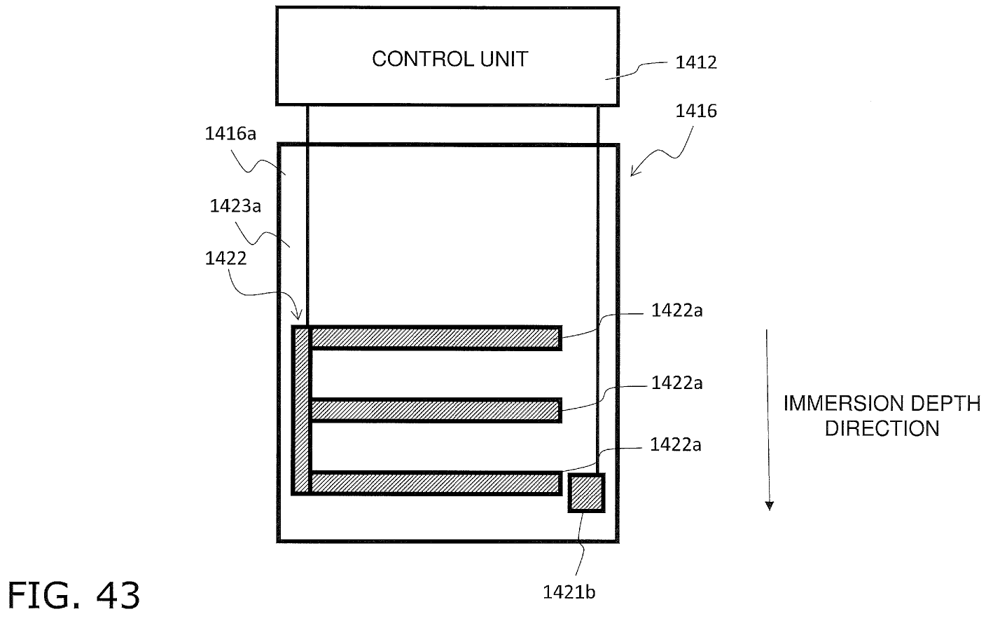

The configuration of a sensor 1416 according to yet another embodiment of the present invention will now be described with reference to FIGS. 43 to 45.

In this embodiment, the immersion detection electrode unit 1422 is a comb shape that includes a plurality of comb teeth 1422a, and therefore differs from the above Embodiments 2 to 5 in which the electrodes 1022a, 1022b, etc., having a simple shape (substantially rectangular) are used.

That is, as shown in FIG. 43, the sensor 1416 of this embodiment comprises a substantially E-shaped immersion detection electrode unit 1422 including a plurality of comb teeth 1422a that extend in a direction substantially perpendicular to the immersion depth direction (substantially the horizontal direction) on the first surface 1423a of the main body 1416a.

Since these comb teeth 1422a extend along substantially the horizontal direction in a state of being installed in the well 8, they are installed substantially parallel to the surface level of the medium L in the well 8.

Therefore, whenever the surface level of the medium L becomes lower than that of the comb teeth 1422a, the current value detected by the current meter when a second voltage is applied between the working electrode 1421a and the immersion detection electrode unit 1422 will change with a large slope.

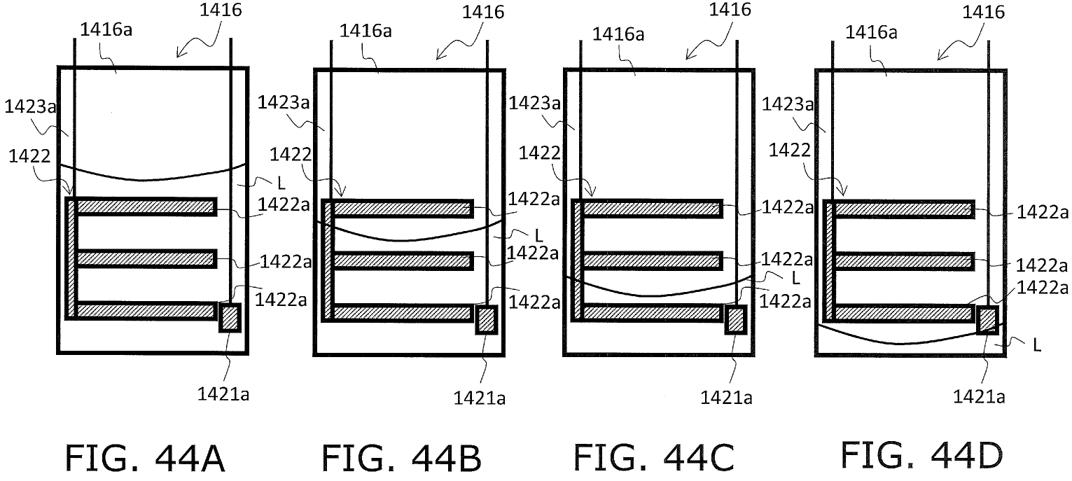

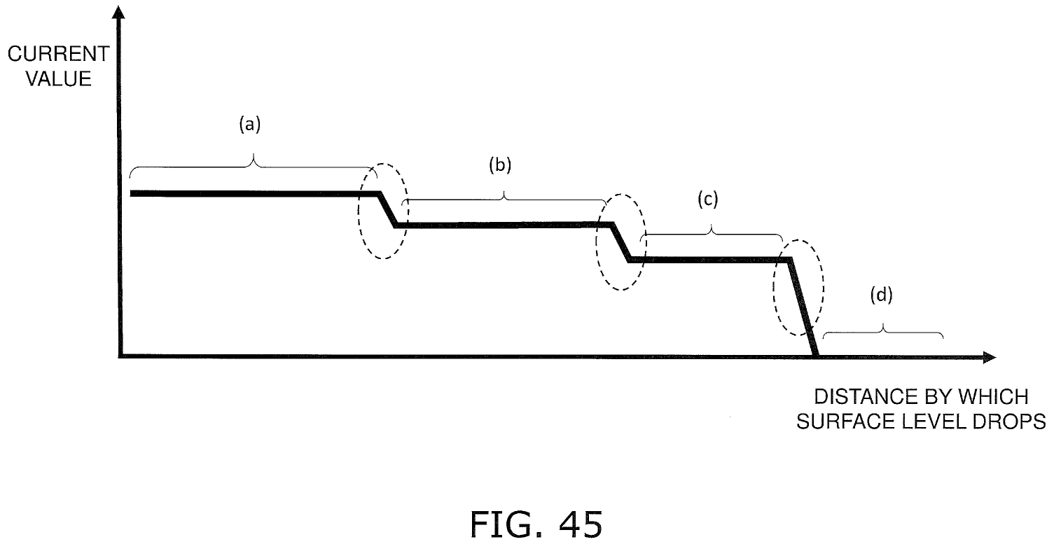

For example, when there is a change from a state in which the surface level is higher than the three comb teeth 1422a shown in FIG. 44A to a state in which the surface level is lower than the comb tooth 1422a disposed at the highest position shown in FIG. 44B, or to a state in which the surface level is lower than the middle comb tooth 1422a shown in FIG. 44C, or to a state in which the surface level is lower than the comb tooth 1422a disposed at the highest position shown in shown in FIG. 44D, then as shown in FIG. 45, whenever the surface level goes past the three comb teeth 1422a, the detected current value will be smaller and have a large slope (the portion surrounded by the broken line in FIG. 45).

Here, parts (a) to (d) in FIG. 45 correspond to FIGS. 44A to 44D.

Consequently, when the immersion detection electrode unit 1422 has a shape including the plurality of comb teeth 1422a, not only the detection of the immersion of the measurement electrode unit, but also the detection of the surface level of the medium L can be performed.

### Embodiment 7

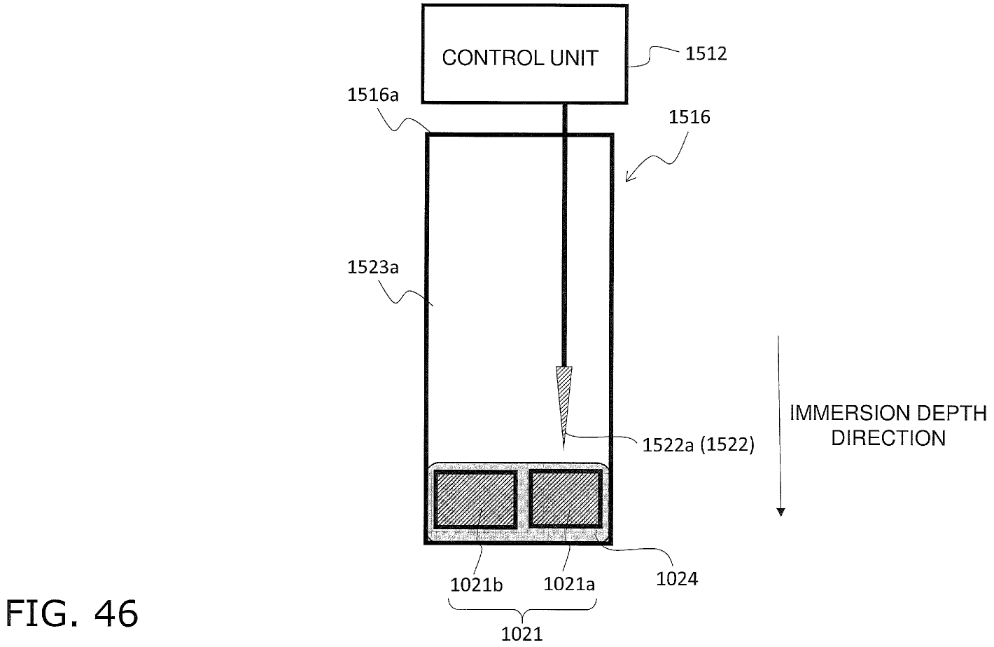

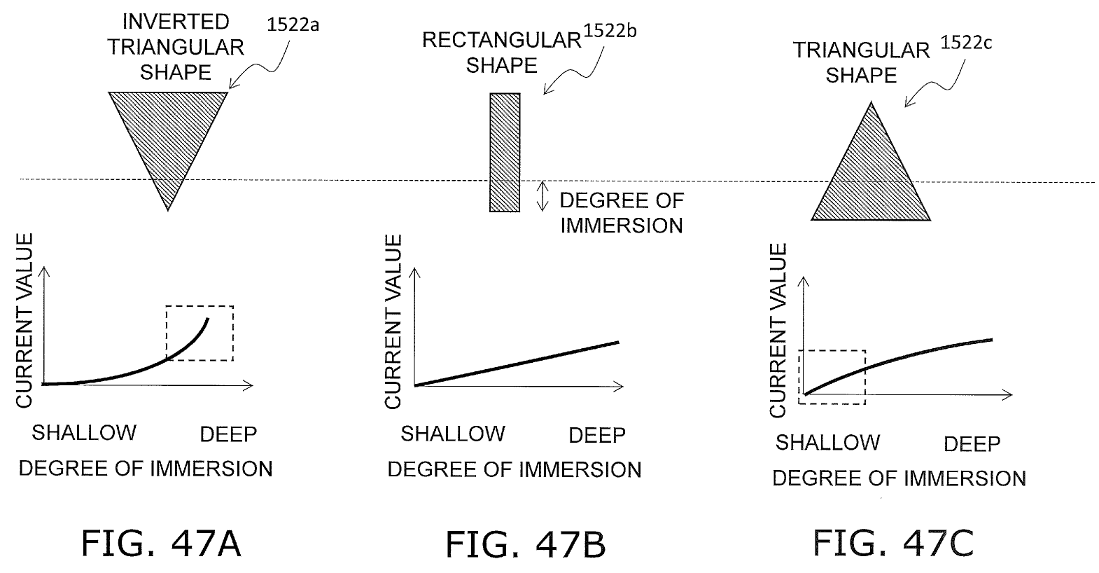

The configuration of a sensor 1516 according to yet another embodiment of the present invention will now be described with reference to FIGS. 46 and 47.

In this embodiment, the electrode 1522a of the immersion detection electrode unit 1522 has a downward-facing triangular shape, which differs from the above Embodiments 2 to 6 in which the electrodes 22a and 22b, etc., have a simple shape (substantially rectangular).

That is, as shown in FIG. 46, the sensor 1516 of this embodiment comprises the electrode 1522a having a triangular shape, whose apex is disposed facing downward, directly above the working electrode 1021a of the measurement-use electrode unit 1021 provided near the lower end portion of the first surface 1523a of the main body 1516a.

Since the electrode 1522a has a long triangular shape in the immersion depth direction, the dimension in the width direction changes in the immersion depth direction. That is, the surface area of the electrode 1522a immersed in the medium L increases along a quadratic curve as the degree of immersion increases.

Therefore, amount of change in the current value detected by the electrode 1522a whose width changes in the immersion depth direction can be amplified according to the change in the surface level of the medium L.

That is, as shown in FIG. 47A, when the inverted triangular electrode 1522a shown in FIG. 46 is used, the change in the detected current value can be increased as the depth of the surface level increases (as the degree of immersion increases).

On the other hand, as shown in FIG. 47B, when the rectangular electrode 1522b of Embodiment 1, etc., described above is used, the change in the depth of the surface level is substantially proportional to the change in the current value.

Also, as shown in FIG. 47C, when the triangular electrode 1522c facing in the opposite direction from that in FIG. 46 (the apex faces upward) is used, the less is the surface level depth, the greater will be the change in the detected current value.

As a result, a change in the surface level at the desired depth can be detected more precisely by selecting the shape of the electrodes of the immersion detection electrode unit, taking into account the position of the immersion depth to be detected and so forth.

### Embodiment 8

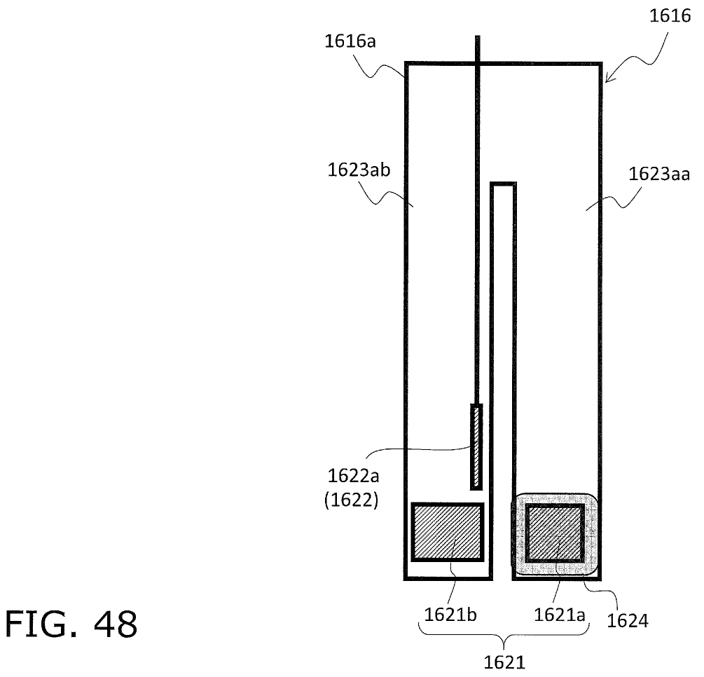

The configuration of the sensor 1616 according to yet another embodiment of the present invention will now be described with reference to FIG. 48.

This embodiment differs from the above Embodiments 2 to 7 in that the lower portion of the main body 1616a of the sensor 1616 is divided into two forks, the working electrode 1621a of the measurement-use electrode unit 1621 is covered by the protective film 1624 on the first surface 1623aa of one of the two forks, and the counter electrode 1621b of the electrode unit 1621 and the electrode 1622a of the immersion detection electrode unit 1622 are provided on the other first surface 1623ab.

That is, in the sensor 1616 of this embodiment, as shown in FIG. 48, the working electrode 1621a and the counter electrode 1621b constituting the measurement-use electrode unit 1021 are provided at separated positions on the first surface 1623aa and 1623ab of the main body 1616a.

Here, when the immersion detection electrode unit 1622 is used to detect the immersion state of the measurement-use electrode unit 1621, a specific voltage (second voltage) may be applied between the electrode 1622a and the counter electrode 1621b, and the current flowing therebetween measured.

Consequently, the electrodes used for immersion detection (the electrode 1622a and the counter electrode 1621b) are disposed at positions separated from the working electrode 1621a, which needs to be covered by the protective film 1624. Therefore, the electrodes used for immersion detection (the electrode 1622a and the counter electrode 1621b) are not covered by the protective film 1624.

As a result, the surface area of the electrodes used for immersion detection can be increased, so measurement sensitivity can be increased. Also, during manufacture, even if the protective film 1624 is formed so as to cover only the working electrode 1621a, the manufacturing can still be easily performed.

### Embodiment 9

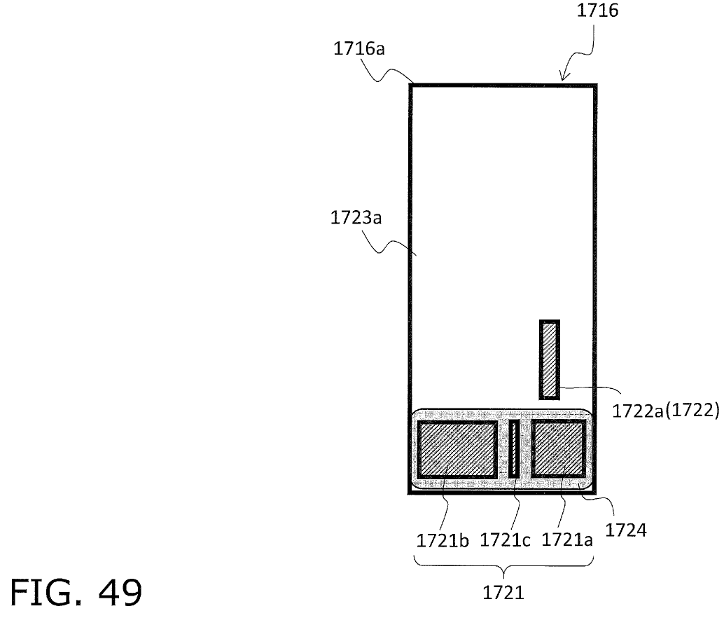

The arrangement of the sensors 1716 according to yet another embodiment of the present invention will now be described with reference to FIG. 49.

In this embodiment, a three-pole configuration including the working electrode 1721a, the counter electrode 1721b, and the reference electrode 1721c is employed as the measurement-use electrode unit 1721, and differs in this respect from the above Embodiments 2 to 8 in which a two-pole configuration including a working electrode and a counter electrode is employed as the measurement-use electrode.

That is, as shown in FIG. 49, the sensor 1716 of this embodiment comprises a working electrode 1721a, a counter electrode 1721b, and a reference electrode 1721c as a measurement-use electrode unit 1721 at the lower end portion of the first surface 1723a of the main body 1716a.

The working electrode 1721a, the counter electrode 1721b, and the reference electrode 1721c are covered by the protective film 1724. The protective film 1724 may be provided so as to cover only the working electrode 1721a as in the embodiments described above.

Furthermore, the electrode 1722a of the immersion detection electrode unit 1722 is disposed directly above the working electrode 1721a.

Consequently, in the detection of the immersion state of the measurement-use electrode unit 1721, a specific voltage (second voltage) is applied between the electrode 1722a of the immersion detection electrode unit 1722 and the working electrode 1721a or the counter electrode 1721b or the reference electrode 1721c, and the current flowing therebetween is measured.

Consequently, the effect obtained with the present invention as described above can also be obtained with the sensor 1716 including the measurement-use electrode unit 1721 having a three-pole configuration including the working electrode 1721a, the counter electrode 1721b, and the reference electrode 1721c.

### Embodiment 10

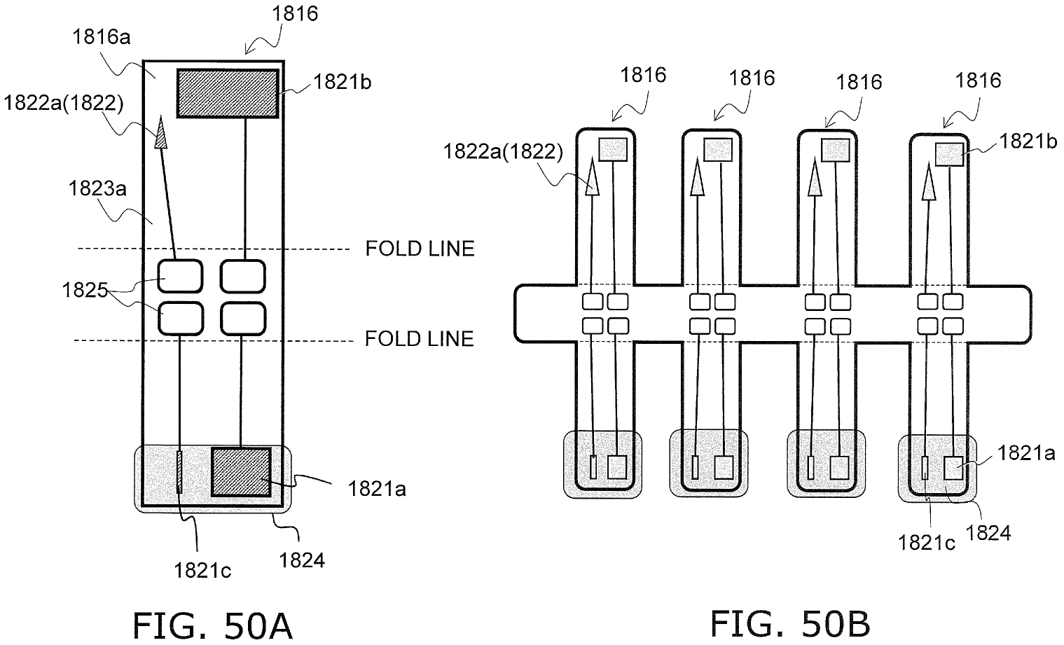

The configuration of the sensor 1816 according to yet another embodiment of the present invention will now be described with reference to FIGS. 50A and 50B.

This embodiment differs from the above Embodiments 2 to 9 in that the sensor 1816 is used in a state of being bent in two places, wherein a working electrode 1821a and a reference electrode 1821c constituting a measurement-use electrode unit are disposed at one end (first end) side in the lengthwise direction of the first surface 1823a of the main body 1816a, and a counter electrode 1821b and an immersion detection electrode unit 1822 (electrode 1822a) are disposed at the other end (second end).

That is, as shown in FIG. 50A, the sensor 1816 of this embodiment is bent into an approximate U shape at two places near the center in the lengthwise direction of the main body 1816a. A working electrode 1821a, a reference electrode 1821c, a counter electrode 1821b, and an immersion detection electrode unit 1822 (electrode 1822a) constituting a measurement-use electrode unit are disposed at the two ends of the main body 1816a in the lengthwise direction.

Connection pads 1825 that are connected to the electrical circuit of the control unit 1012 is provided between the two fold lines.

Consequently, the electrodes used for immersion detection (the electrode 1822a and the counter electrode 1821b) are disposed at positions separated from the working electrode 1821a, which needs to be covered by the protective film 1824. Therefore, the electrodes used for immersion detection (electrode 1822a and counter electrode 1821b) are not covered by the protective film 1824.

As a result, the surface area of the electrodes used for immersion detection can be increased, so measurement sensitivity can be increased. Also, during manufacture, even if the protective film 1824 is formed so as to cover only the working electrode 1821a, the manufacturing can still be easily performed.

In addition, a plurality of the sensors 1816 shown in FIG. 50A may be connected to form a unitized configuration as shown in FIG. 50B.

In this case, a plurality of sensors can be provided for a single substrate. Furthermore, even when the protective film 1824 is provided so as to cover the working electrode 1821a and the reference electrode 1821c, the protective film 1824 covering the working electrode 1821a, etc., grouped together on one end side can be easily formed, so manufacturing is easier.

### Embodiment 11

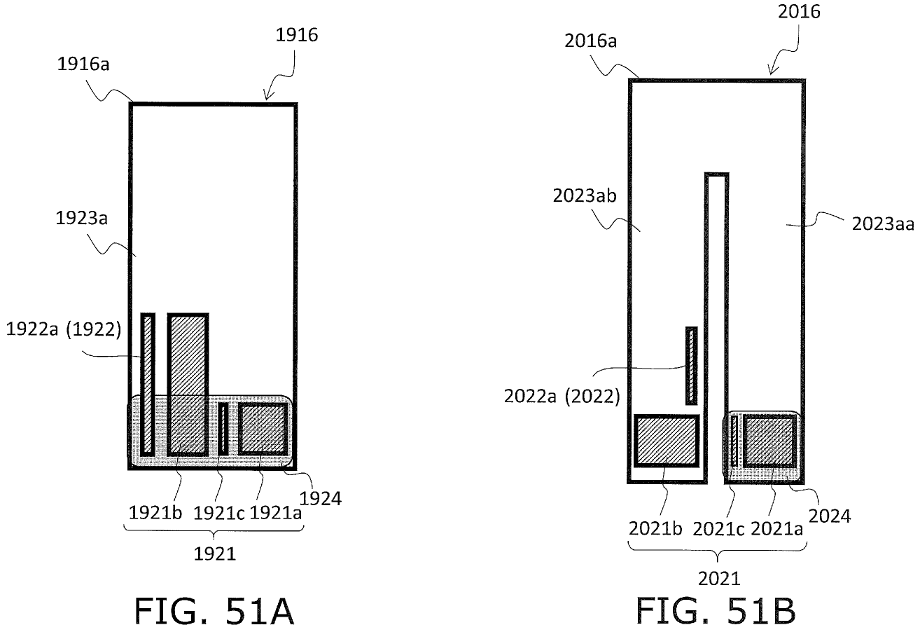

The configuration of the sensor 1916 according to yet another embodiment of the present invention will now be described with reference to FIG. 51A.

This embodiment differs from the above Embodiments 2 to 10 in that a counter electrode 1921b constituting a measurement-use electrode unit 1921 is provided so as to extend above a working electrode 1921a and a reference electrode 1921c.

This embodiment also differs from Embodiments 2 to 10 in that an electrode 1922a of an immersion detection electrode unit 1922 is provided so as to extend from a height position substantially equal to that of the measurement-use electrode unit 1921 to above the working electrode 1921a of the electrode unit 1921, etc.

That is, in the sensor 1916 of this embodiment, as shown in FIG. 51A, four electrodes (the working electrode 1921a, the counter electrode 1921b, the reference electrode 1921c, and the electrode 1922a) are provided near the lower end portion of the main body 1916a. The counter electrode 1921b and the electrode 1922a are disposed so as to extend to a position higher than the working electrode 1921a and the reference electrode 1921c.

Consequently, by applying a specific voltage (second voltage) between the electrode 1922a and the counter electrode 1921b and measuring the current flowing between them, it is easy to detect whether or not the working electrode 1921a is in an immersed state.

In particular, in this embodiment, the electrode 1922a of the immersion detection electrode unit 1922 has an elongated shape in the immersion depth direction. Therefore, when the surface level of the medium L changes, the surface area of the electrode 1922a immersed in the medium L also changes, so the magnitude of the detected current value also changes.

Consequently, measuring the current value allows not only the immersion state of the working electrode 1921a to be detected, but also the surface level of the medium L.

In the sensor 1916 of this embodiment, the protective film 1924 is formed so as to cover the vicinity of the lower end portion of the main body 1923a. More precisely, the protective film 1924 is formed to cover the entire working electrode 1021a and reference electrode 1921c, and to cover the lower half of the counter electrode 1921b and the electrode 1922a.

Consequently, immersion detection can be carried out more efficiently by utilizing the upper half regions of the electrode 1922a and the counter electrode 1921b, which perform immersion detection, that are not covered by the protective film 1924.

### Embodiment 12

The configuration of the sensor 2016 according to yet another embodiment of the present invention will now be described with reference to FIG. 51B.

In this embodiment, the lower part of the main body 2016a is divided into two forks, the working electrode 1021a and the reference electrode 1021c of the measurement-use electrode unit 2021 are provided in a state of being covered by the protective film 1024 on a first surface 2023aa which (one of the two forks), and the counter electrode 2021b of the electrode unit 2021 and the electrode 2022a of the immersion detection electrode unit 2022 are provided on the other first surface 2023ab, and in this respect differs from the above the sensor 1616 of Embodiment 8 in which no reference electrode is included.

That is, with the sensor 2016 of this embodiment, as shown in FIG. 51B, the reference electrode 2021c is provided on the first surface 2023aa on the side where the working electrode 2021a is provided, and the working electrode 2021a and the reference electrode 2021c are covered by a protective film 2024.

Consequently, since the counter electrode 2021b and the electrode 2022a used for immersion detection are not covered by the protective film, there is no need for a waiting time until the components of the medium permeate through the protective film, and the processing speed of immersion detection can be improved.

Also, since the working electrode 2021a that requires the protective film 2024 and the counter electrode 2021b side that does not require the protective film are provided at positions separated from each other on the main body 2016a, the manufacturing process can be simplified in the formation of the protective film 2020 so as to cover the working electrode 2021a.

### Embodiment 13

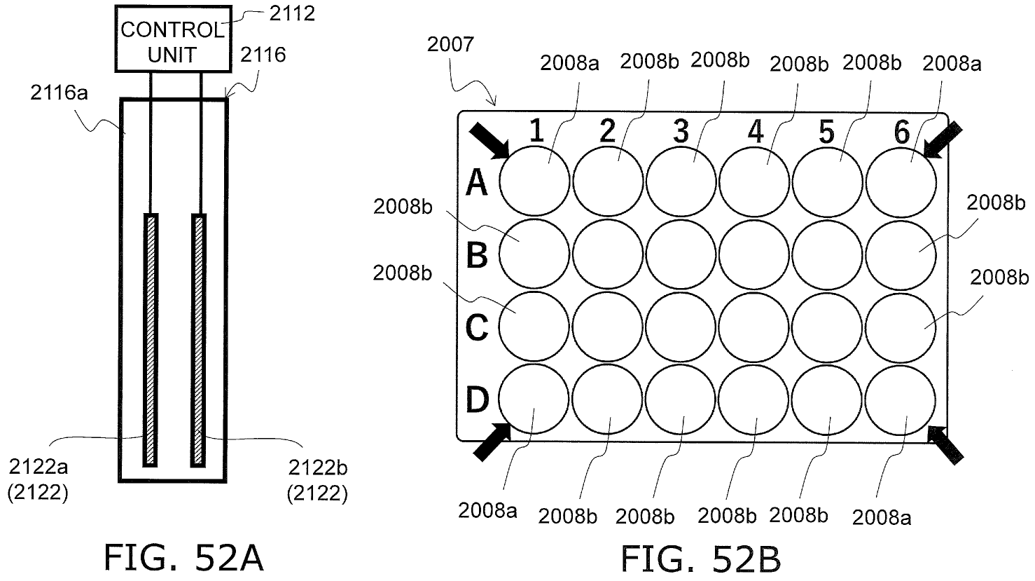

The configuration of the sensor 2116 according to yet another embodiment of the present invention will now be described with reference to FIGS. 52A and 52B.

In this embodiment, two electrodes 2122a and 2122b that are connected to the control unit 2112 and provided in the main body 2116a are used as the immersion detection electrode unit 2122, and in this respect differs from the above Embodiments 2 to 12 in which the sensor configuration includes a measurement-use electrode unit.

That is, as shown in FIG. 52A, the sensor 2116 of this embodiment comprises the immersion detection electrode unit 2122 including the two immersion detection electrodes 2122a and 2122b, and is configured as a sensor that only performs immersion detection.

As shown in FIG. 52B, the sensors 2116 are disposed in the wells 2008a disposed at the four corners of the culture container 2007, which includes a total of 24 wells disposed in a substantially quadrangular (rectangular) shape measuring 4 wells high × 6 wells wide.

That is, in this embodiment, the sensors 2116 shown in FIG. 52A are installed in the wells 2008a disposed at the four corners shown in FIG. 52B, and the sensors 16, etc., according to the various above-mentioned embodiments, or dedicated measurement sensors having a measurement-use electrode unit 21 but not having an immersion detection electrode unit are installed in the wells 2008b other than those at the four corners.

Here, since the wells 2008a disposed at the four corners where the sensors 2116 are installed have a larger surface area in contact with the outside air, the medium L or other such liquid sample contained therein is more likely to evaporate. That is, the liquid sample evaporates faster and the surface level is more likely to drop in the wells 2008a than in the other wells 2008b.

Therefore, installing the sensors 2116 including the immersion detection electrode unit 2122 in the wells 2008a disposed at the four corners, where the surface level of the liquid sample tends to be the lowest, makes it possible to determine whether the measurement electrode units of the sensors installed in the other wells are sufficiently immersed.

In this embodiment, as shown in FIG. 52B, sensors for immersion detection are installed in the wells disposed at the four corners our of the plurality of wells disposed in a substantially quadrangular shape, but if a plurality of wells are disposed in a substantially circular shape, for example, sensors for immersion detection may be installed in the wells disposed on the outermost peripheral side where the liquid sample is likely to evaporate.

### Other Embodiments

Embodiments of the present invention were described above, but the present invention is not limited to or by the above embodiments, and various modifications are possible without departing from the gist of the invention.

### A

In the above embodiments, an example was given in which the portion of the main body 16a of the sensor 16 serving as the liquid holding portion 22 on the first surface 23a was mainly provided on the upper portion of the electrode unit 21. However, the present invention is not limited to this.

For instance, the configuration may be such that the liquid holding portion is provided from the side to above the electrode unit on the first surface.

### B

In the above embodiments, an example was given in which the sensor 16 had the main body 16a having a substantially plate-like shape. However, the present invention is not limited to this.

For instance, the shape of the main body of the sensor is not limited to being substantially plate shaped, and may instead be some other shape, such as a substantially cuboid shape.

### C

In the above embodiments, an example was given in which the sensor 16 was substantially L-shaped. However, the present invention is not limited to this.

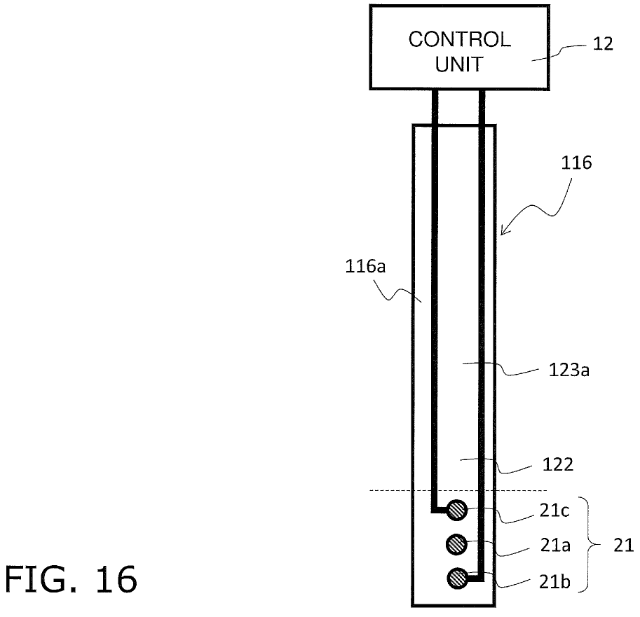

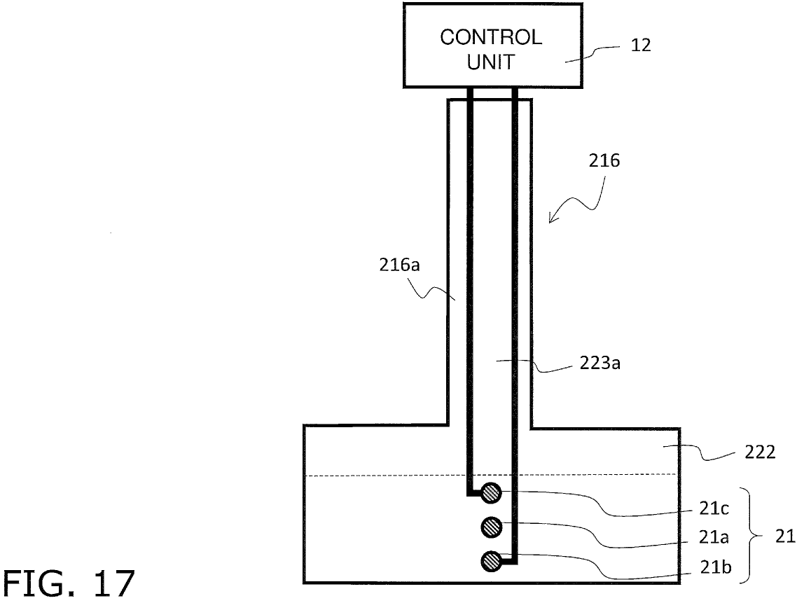

For instance, the substantially I-shaped sensor 116 shown in FIG. 16 may be used, or the substantially inverted T-shaped sensor 216 shown in FIG. 17 may be used.

In the case of the substantially I-shaped sensor 116 shown in FIG. 16, the configuration may be such that the above-mentioned electrode unit 21 is provided in the lower end region of the first surface 123a of the rectangular main body 116a when seen in front view, and the liquid holding portion 122 is provided in the upper region of the electrode unit 21.

In the case of the substantially inverted T-shaped sensor 216 shown in FIG. 17, the configuration may be such that the above-mentioned electrode unit 21 is provided in the wide lower end region of the first surface 223a of the inverted T-shaped main body 216a when seen in front view, and the liquid holding portion 222 is provided in the upper region of the electrode unit 21.

### D

In the above embodiments, an example was given in which the distance between the first surface 23a and the inner peripheral surface 8a of the well 8 capable of effectively holding the liquid sample (medium) in the liquid holding portion 22, that is, the proximity where surface tension is generated in the liquid sample, was a distance of 1 to 2 mm. However, the present invention is not limited to this.

For instance, the distance at which the liquid sample can be effectively held in the liquid holding portion may be larger or smaller than 1 to 2 mm, depending on the type of the liquid sample, etc.

### E

In the above embodiments, an example was given in which one sensor 16 was installed in one well 8 included in the culture container 7. However, the present invention is not limited to this.

For instance, the configuration may be such that two or more sensors are installed in one well (container). In this case, the inside diameter of the well (container) may be increased and a plurality of sensors installed in the vicinity of the inner peripheral surface.

### F

In the above embodiments, an example was given in which the sensors 16 were immersed in containers (wells 8) having a substantially circular shape (substantially cylindrical shape) in top view, and the cell culture environment of the medium was analyzed. However, the present invention is not limited to this.

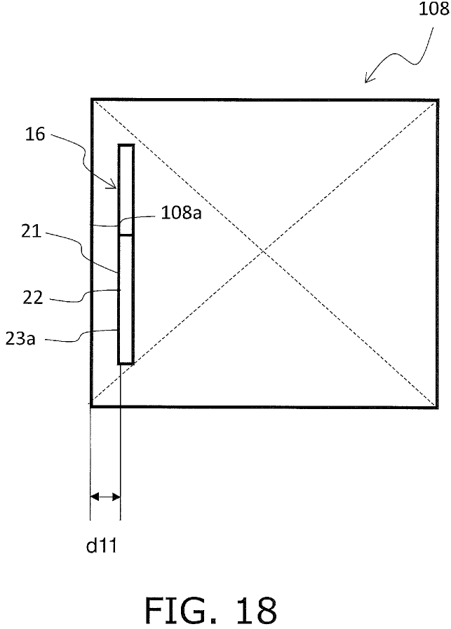

For instance, the container in which the sensor is immersed is not limited to a substantially cylindrical container, and may instead be the container 108 having a substantially rectangular shape (such as a substantially square shape) in the top view shown in FIG. 18.

In this case, it is preferable to use a sensor 16 in which the width of the portion provided with the electrode unit is less than the length of a diagonal line of the substantially square container 108 in top view.

Consequently, the sensor 16 can be disposed so that the first surface 23a is close to the inner wall surface 108a of the substantially rectangular container 108.

### G

In the above embodiments, an example was given in which the electrode unit 21 to which voltage was applied included the working electrode 21a, the counter electrode 21b, and the reference electrode 21c. However, the present invention is not limited to this.

For instance, the type of electrode constituting the electrode unit is not limited to the configuration of the above embodiments, and other types of electrodes may be provided instead.

### H

In the above embodiments, an example was given in which a plurality of sensors 16 were formed by cutting out parts of a single substrate 13. However, the present invention is not limited to this.

For instance, the configuration may be such that one sensor is provided on the substrate, or the configuration may be such that the substrate and the sensor are connected by adhesive bonding, welding, or some other such means.

### I

In the above embodiments, an example was given in which a medium for performing cell culture was used as the liquid sample. However, the present invention is not limited to this.

For instance, the liquid sample is not limited to a medium for performing cell culture, and may instead be a liquid sample that will be analyzed, etc.

### J

In the above embodiments, an example was given in which all the electrodes constituting the electrode unit 21 (working electrode 21a, counter electrode 21b, reference electrode 21c) were provided on the first surface 23a of the main body 16a of the sensor 16. However, the present invention is not limited to this.

For instance, the configuration may be such that among the electrodes constituting the electrode unit, only the working electrode is provided on the first surface, and the counter electrode and the reference electrode are provided on the second surface.

That is, the configuration may be such that at least one of the electrodes constituting the electrode unit is disposed.

### K

In Embodiment 2 above, an example was given in which an electrode unit 1021 for measuring a liquid sample and an immersion detection electrode unit 1022 were provided on the first surface 1023a of the main body 1016a of the sensor 1016. However, the present invention is not limited to this.

For instance, the configuration may be such that the measurement electrode unit for measuring the liquid sample and the immersion detection electrode unit for detecting the immersion state of the measurement electrode unit are provided on different surfaces of the main body of the sensor.

### L

In Embodiment 2 above, an example was given in which all the electrodes constituting the electrode unit 1021 (working electrode 1021a and counter electrode 1021b) were provided on the first surface 1023a of the main body 1016a of the sensor 1016. However, the present invention is not limited to this.

For instance, the configuration may be such that among the electrodes constituting the electrode unit, only the working electrode is provided on the first surface, and the counter electrode and the reference electrode are provided on the second surface.

That is, the configuration may be such that at least one of the electrodes constituting the electrode unit is disposed.

### M

In Embodiment 2 above, an example was given in which, in measuring the medium (liquid sample), a voltage was applied to the immersion detection electrode unit 1022 in order to detect the immersion state of the measurement electrode unit 1021, and then the application of voltage was temporarily stopped, after which voltage was applied to the measurement-use electrode unit 1021. However, the present invention is not limited to this.

For instance, the immersion detection and the measurement may be performed at the same time without providing any voltage non-application period between the immersion detection and the measurement.

### N

In Embodiment 2 above, an example was given in which the sensor 1016 was disposed near the approximate center of the well 1008 to detect the immersion state and measure. However, the present invention is not limited to this.

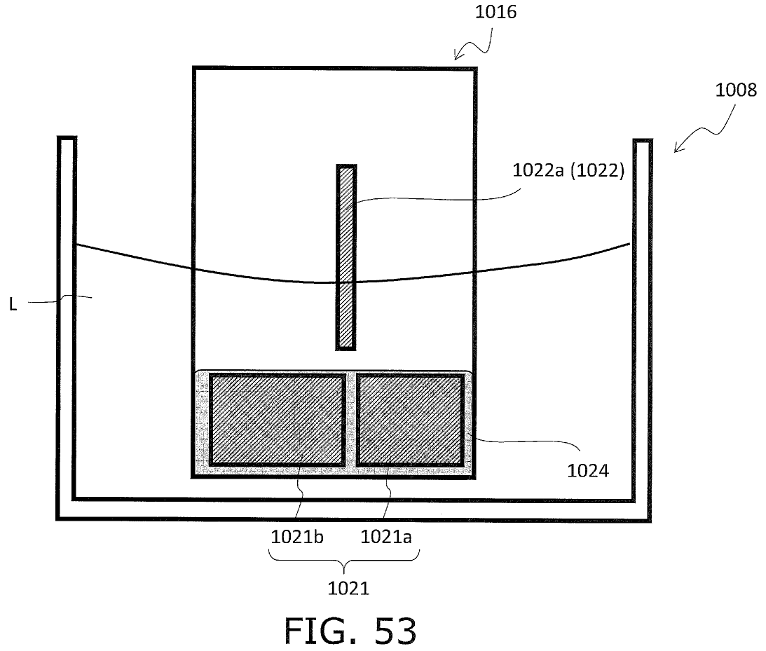

For instance, as shown in FIG. 53, the sensor 16 may be installed so that the electrode 1022a of the immersion detection electrode unit 1022 provided to the main body 1016a of the sensor 1016 is disposed in the approximate center of the well 1008.

In this case, the electrode 1022a of the immersion detection electrode unit 1022 is disposed near the approximate center of the well 1008, where the surface level of the medium L tends to be the lowest due to the above-mentioned meniscus effect. Therefore, when it is detected that the electrode 1022a is in the immersed state, it can be safely concluded that the measurement-use electrode unit 1021 disposed below the electrode 1022a is in an immersed state.

As a result, the immersion state of the measurement-use electrode unit can be detected more reliably, and the liquid sample can be measured more accurately.

### O

In Embodiment 6 above, an example was given in which a sensor 1416 comprising a substantially E-shaped immersion detection electrode unit 1422 including a plurality of comb teeth 1442a extending in a direction substantially perpendicular to the immersion depth direction (substantially horizontal direction) is provided on the first surface 1423a of the main body 1416a as shown in FIG. 43 in order to detect the surface level in addition to detecting the immersion state. However, the present invention is not limited to this.

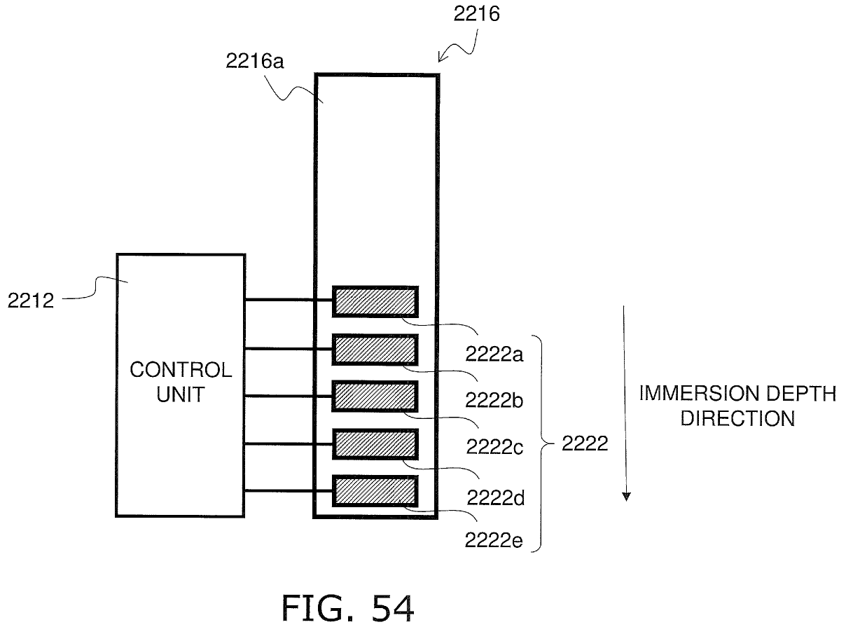

For instance, as shown in FIG. 54, the control unit 2212 may digitally detect the surface level of the medium contained in the well by using the immersion detection electrode unit 2222 including a plurality of electrodes 21222a to 2222e disposed along the immersion depth direction.

### P

In Embodiment 2, etc., above, an example was given in which the main body 1016a of the sensor 1016 was substantially I-shaped. However, the present invention is not limited to this.

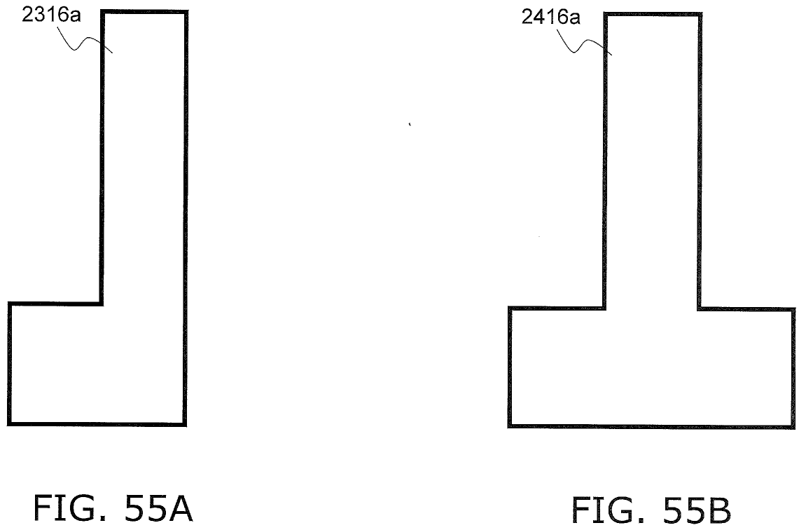

For instance, the sensor having a substantially L-shaped main body 2316a shown in FIG. 55A may be used, or the sensor having a substantially inverted T-shaped main body 2416a shown in FIG. 55B may be used.

### INDUSTRIAL APPLICABILITY

Because the sensor of the present invention has the effect of improving the measurement accuracy of a sensor used in a state of being immersed in a liquid sample, it can be broadly applied to various analysis devices that make use of sensors.

### REFERENCE SIGNS LIST

1 cell culture device
2 culture chamber
3 cell culture analysis device
4 door
5 main body case
6 culture container installation unit
6a recess
7 culture container
8 well (container)
8a inner peripheral surface (inner wall surface)
9 sensor unit
10 leg
11 positioning hole
12 control unit
13 substrate
14 bottom cover
15 top cover
16 sensor
16a main body
17 bent portion
18 L-shaped part
18a vertical side cutout portion
18b horizontal side cutout portion
19 wiring
20a, 20b connection portion
21 electrode unit
21a working electrode
21b counter electrode
21c reference electrode
22 liquid holding unit
23a first surface
23b second surface
30 through-hole
31 support portion
32 pressing portion
33 measurement unit
34 control unit
35 storage unit
36 communication unit
37 external device
38 communication unit
39 control unit
40 display unit
41 input unit
108 container
108a inner wall surface
116 sensor
116a main body
122 liquid holding unit
123a first surface
216 sensor
216a main body
222 liquid holding unit
223a first surface
1006 culture container installation unit
1006a recess
1007 culture container
1008 well (container)
1008a inner peripheral surface
1009 sensor unit
1010 leg
1011 positioning hole
1012 control unit
1012a voltage application unit
1012b current meter
1012ca switch
1012cc switch
1012da D/A converter
1012db D/A converter
1012e A/D converter
1013 substrate
1014 bottom cover
1015 top cover
1016 sensor
1016a main body
1017 bent portion
1018 substantially I-shaped portion
1018a vertical side cutout portion
18b horizontal side cutout portion
1019 wiring
1020a, 1020b connection portion
1021 electrode unit (measurement electrode unit)
1021a working electrode
1021b opposite electrode
1022 immersion detection electrode unit
1022a first electrode
1022b second electrode
1023a first surface
1024 protective film
1030 through-hole
1031 support portion
1032 pressing portion
1033 measurement unit
1034 control unit
1035 storage unit
1036 communication unit
1037 immersion detection unit
1038 display unit
1040 external device
1041 communication unit
1042 control unit
1043 display unit
1044 input unit
1112 control unit
1112a selection circuit
1116 sensor
1116a main body
1122 immersion detection electrode unit
1122a first electrode
1122b second electrode
1122c third electrode
1123a first surface
1134 control unit
1212 control unit
1216 sensor
1216a main body
1222 immersion detection electrode unit
1222a first electrode
1223a first surface
1234 control unit
1312 control unit
1312c counter electrode-side circuit
1316 sensor
1316a main body
1322 immersion detection electrode unit
1322a first electrode
1323a first surface
1324 protective film
1334 control unit
1416 sensor
1416a main body
1421a working electrode
1422 immersion detection electrode unit
1422a comb teeth
1423a first surface
1516 sensor
1516a main body
1522 immersion detection electrode unit
1522a electrode
1522b electrode
1522c electrode
1523a first surface
1616 sensor
1616a main body
1621 electrode unit (measurement electrode unit)
1621a working electrode
1621b counter electrode
1622 immersion detection electrode unit
1622a electrode
1623aa, 1623ab first surface
1624 protective film
1716 sensor
1716a main body
1721 electrode unit (measurement electrode unit)
1721a working electrode
1721b counter electrode
1721c reference electrode
1722 immersion detection electrode unit
1722a electrode
1723a first surface
1724 protective film
1816 sensor
1816a main body
1821a working electrode
1821b counter electrode
1821c reference electrode
1822 immersion detection electrode unit
1822a electrode
1823a first surface
1824 protective film
1825 connection pad
1916 sensor
1916a main body
1921 electrode unit (measurement electrode unit)
1921a working electrode
1921b counter electrode
1921c reference electrode
1922 immersion detection electrode unit
1922a electrode
1923a first surface
1924 protective film
2007 culture container
2008a, 2008b well (container)
2016 sensor
2016a main body
2021 electrode unit (measurement electrode unit)
2021a working electrode
2021b counter electrode
2021c reference electrode
2022 immersion detection electrode unit
2022a electrode
2023aa, 2023ab first surface
2024 protective film
2112 control unit
2116 sensor
2116a main body
2122 immersion detection electrode unit
2122a electrode
2122b electrode
2212 control unit
2222 immersion detection electrode unit
2222a-2222e electrodes
2316a main body
2416a main body
d1, d2 distance
L medium
O center

1. A sensor that is used in a state of being immersed in a liquid sample inside a container, and that measures the liquid sample, the sensor comprising:
a main body having a first surface and a second surface that is on an opposite side from the first surface;
an electrode unit that is provided on the first surface of the main body and to which a specific voltage is applied during measurement in a state of being immersed in the liquid sample; and
a liquid holding portion that is provided around the electrode unit on the first surface, that is disposed near an inner wall surface of the container, and that holds the liquid sample up to above the electrode unit between the first surface and the inner wall surface.

2. The sensor according to claim 1,
wherein the liquid holding portion holds the liquid sample on a side of the first surface up to a position higher than on a side of the second surface when the electrode unit is immersed in the liquid sample.

3. The sensor according to claim 1,
wherein the liquid holding portion is provided to an upper portion of the electrode unit on the first surface in a state in which the electrode unit is immersed in the liquid sample.

4. The sensor according to claim 1,
wherein the liquid holding portion has substantially the same width as the first surface, or has a width that is greater than the width of the first surface, in the portion where the electrode unit is provided.

5. The sensor according to claim 1,
wherein the main body is disposed near the inner wall surface up to a distance at which surface tension is generated in the liquid sample held between the first surface and the inner wall surface of the container.

6. The sensor according to claim 5,
wherein the container is substantially circular in top view, and
the first surface is disposed at a position on a chord of the substantially circular shape with respect to the inner wall surface of the container that is substantially circular in top view.

7. The sensor according to claim 6,
wherein a width of the first surface provided with the electrode unit is less than a diameter of the substantially circular container.

8. The sensor according to claim 5,
wherein the container is substantially rectangular in top view, and
the first surface is disposed so as to be near one side of the inner wall surface of the container that is substantially rectangular in top view.

9. The sensor according to claim 8,
wherein a width of the first surface provided with the electrode unit is less than a diagonal length of the substantially rectangular container.

10. The sensor according to claim 1,
wherein the electrode unit includes at least one of a reference electrode, a working electrode, and a counter electrode.

11. The sensor according to claim 1,
wherein the main body is substantially L-shaped or substantially inverted T-shaped in front view.

12. A sensor unit, comprising:
the sensor according to claim 1;
a substrate provided with a plurality of the sensors; and
a connection portion that connects the substrate and the sensor.

13. The sensor unit according to claim 12,
wherein the plurality of sensors are formed by cutting out a part of the substrate.

14. The sensor unit according to claim 12,
further comprising a bottom cover provided below the substrate, and a top cover provided above the substrate,
wherein the substrate is sandwiched between the bottom cover and the top cover from above and below.

15. The sensor unit according to claim 14,
wherein the bottom cover has through-holes through which the sensors pass downward.

16. A cell culture analysis device, comprising:
the sensor unit according to claim 12; and
a culture container installation unit on which the sensor unit and the container containing the liquid sample are placed.

17. A sensor that is used in a state of being immersed in a liquid sample inside a container, and that is configured to measure the liquid sample, the sensor comprising:
a main body having a first surface and a second surface that is on an opposite side from the first surface; and
an electrode unit that is provided on the first surface of the main body and to which a specific voltage is applied during measurement in a state of being immersed in the liquid sample,
wherein, in carrying out a measurement, the main body is installed at a position that is offset from a center of the container.

18. A sensor that is used in a state of being immersed in a liquid sample inside a container, and that is configured to measure the liquid sample, the sensor comprising:
a main body;
a measurement electrode unit that is provided to the main body and to which a specific first voltage is applied during measurement in a state of being immersed in the liquid sample; and
an immersion detection electrode unit that is provided above the measurement electrode unit in the main body in a state of being immersed in the liquid sample, and to which a specific second voltage is applied during detection of whether or not the measurement electrode unit is in a state of being immersed in the liquid sample.

19. The sensor according to claim 18,
wherein the main body has a first surface to which the measurement electrode unit is provided, and
the immersion detection electrode unit is disposed above the measurement electrode unit on the first surface.

20. The sensor according to claim 18,
further comprising a protective film that covers at least a part of the measurement electrode unit.

21. The sensor according to claim 18,
wherein the measurement electrode unit includes two poles, namely, a working electrode and a counter electrode, or includes three poles, namely, a working electrode, a counter electrode, and a reference electrode.

22. The sensor according to claim 20,
wherein the protective film is provided so as to cover at least a working electrode included in the measurement electrode unit.

23. The sensor according to claim 18,
wherein the immersion detection electrode unit is disposed directly on a working electrode included in the measurement electrode unit.

24. The sensor according to claim 18,
wherein the immersion detection electrode unit is installed so as to match a width of a working electrode included in the measurement electrode unit in a substantially horizontal direction.

25. The sensor according to claim 18, wherein the immersion detection electrode unit has two or three electrodes.

26. The sensor according to claim 18,
wherein the immersion detection electrode unit has one electrode, and
the second voltage is applied between one electrode of the immersion detection electrode and at least one of the electrodes included in the measurement electrode unit.

27. The sensor according to claim 18,
wherein the immersion detection electrode unit extends in a form of a plurality of comb-like teeth substantially parallel to a liquid surface level of the liquid sample.

28. The sensor according to claim 18,
wherein the immersion detection electrode unit has a shape whose dimension in a substantially horizontal direction changes in an immersion depth direction in a state of being immersed in the liquid sample.

29. The sensor according to claim 28,
wherein the immersion detection electrode unit has a substantially triangular shape.

30. The sensor according to claim 18,
wherein a working electrode included in the measurement electrode unit is disposed at a position in the main body that is away from a counter electrode included in the measurement electrode unit.

31. The sensor according to claim 18,
wherein the immersion detection electrode unit is disposed in an approximate center portion of the container.

32. A measurement device, comprising:
the sensor according to claim 18;
a voltage application unit configured to apply the specific first voltage and the specific second voltage to the measurement electrode unit and the immersion detection electrode unit; and
a control unit configured to perform measurement on the liquid sample on the basis of a first current value obtained by applying the first voltage to the measurement electrode unit, and detect whether or not the measurement electrode unit is in an immersed state on the basis of a second current value obtained by applying the second voltage to the immersion detection electrode unit.

33. The measurement device according to claim 32,
wherein the control unit detects a liquid surface level of the liquid sample on the basis of the second current value obtained by applying the second voltage to the immersion detection electrode unit.

34. The measurement device according to claim 32,
wherein the voltage application unit applies a substantially AC voltage to the measurement electrode unit and the immersion detection electrode unit.

35. A sensor unit, comprising:
the sensor according to claim 18;
a substrate provided with a plurality of the sensors; and
a connection portion that connects the substrate and the sensors.

36. The sensor unit according to claim 35,
wherein the plurality of sensors are formed by cutting out parts of the substrate.

37. The sensor unit according to claim 35 ,
further comprising a bottom cover provided below the substrate, and a top cover provided above the substrate,
wherein the substrate is configured to be sandwiched between the bottom cover and the top cover from above and below.

38. The sensor unit according to claim 37,
wherein the bottom cover has through-holes through which the sensors pass downward.

39. A sensor unit comprising a plurality of sensors that are used in a state of being immersed in a liquid sample contained in a plurality of containers, the sensor unit further comprising:
a first sensor provided at a position corresponding to a first container disposed on at least one edge, out of the plurality of containers; and
a second sensor provided at a position corresponding to a second container disposed at a position other than that of the first sensor, out of the plurality of containers,
wherein the first sensor has an immersion detection electrode unit configured to detect whether or not a measurement electrode of the second sensor immersed in the liquid sample is itself immersed in the liquid sample, and
the second sensor has a measurement electrode unit configured to measure the liquid sample.

40. The sensor unit according to claim 39,
wherein the first sensors are disposed at positions corresponding to the containers disposed at four corners of a substantially rectangular shape, among a plurality of containers disposed in the substantially rectangular shape.

41. A cell culture analysis device, comprising:
the sensor unit according to claim 35; and
a culture container installation unit on which are placed the sensor unit and the container in which the liquid sample is contained.

42. The cell culture analysis device according to claim 41, further comprising:
an immersion detection unit that is connected to the immersion detection electrode units of the plurality of sensors provided on the substrate, and that is configured to detect an immersion state of the measurement electrode unit with respect to the liquid sample contained in the container; and
a display unit configured to display a detection result from the immersion detection unit.

43. A liquid sample measurement method for performing measurement using the sensor according to claim 18, the method comprising:
an immersion detection step of applying the second voltage to the immersion detection electrode unit; and
a measurement step of applying the first voltage to the measurement electrode unit.

44. The liquid sample measuring method according to claim 43,
further comprising a voltage application stoppage step of stopping an application of the second voltage to the immersion detection electrode unit, in between the immersion detection step and the measurement step.
